(19) United States
(12) Patent Application Publication
(10) Pub. No.: US 2004/0220091 A1
(43) Pub. Date: Nov. 4, 2004

(54) Process for preparation of cyclosporin A analogs

(76) Inventors: Jean-Michel Adam, Reinach (CH); Mark Abel, Edmonton (CA); Seetharaman Jayaraman, Edmonton (CA)

Correspondence Address: HOFFMANN-LA ROCHE INC., PATENT LAW DEPARTMENT, 340 KINGSLAND STREET, NUTLEY, NJ, 07110

(21) Appl. No.: 10/817,991

(22) Filed: Apr. 5, 2004

(30) Foreign Application Priority Data
Apr. 8, 2003 (EP) 03007921.4

(51) Int. Cl. A61K 38/13; C07K 7/64

(52) U.S. Cl. 514/11; 530/317

(57) ABSTRACT

A new process for the preparation of a cyclosporin A analog of formula I

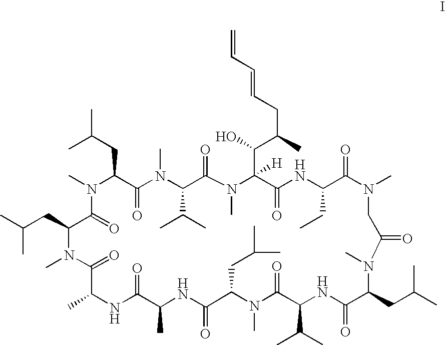

comprising:

a) allylating a protected cyclosporin A aldehyde with a allylmetal reagent and

b) converting the compound obtained in step a) to the cyclosporin A analog of formula I has been identified. Intermediates for this process and processes for the preparation of such intermediates are also discussed.

This invention relates to a new process for the preparation of cyclosporin A analog of formula I:

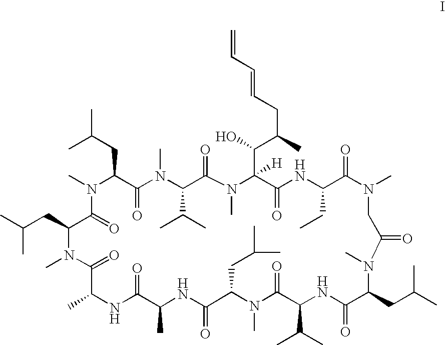

As well as intermediates for this process and processes for the preparation thereof.

The cyclosporin A analog of formula I is structually identical to cyclosporin A except for modification at the 1-amino acid residue. This analog is disclosed in WO 99/18120 and U.S. Provisional Patent Application No. 60/346,201. Hereinafter this analog is mentioned as (E)-ISA247.

Tetrahedron Letters, Vol.22, No.29, p2751-2752, 1981 discloses one of the intermediates of the process of this invention, namely pinacol (E)-1-trimethylsilyl-1-propene-3-boronate, and the allylation process using it.

Tetrahedron Letters, Vol.36, No.10, p1583, 1995 discloses allylation process using tartrate modified (E)-γ-(trimethylsilyl)allylboronate.

### SUMMARY OF THE INVENTION

In a first aspect, this invention provides a process for the preparation of a cyclosporin A analog of formula I

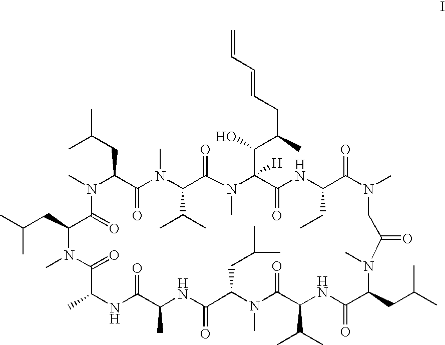

comprising:

a-i) allylating a compound of formula II

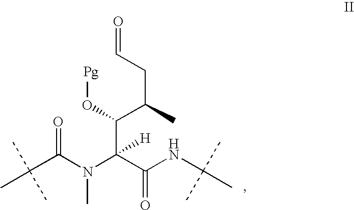

wherein Pg is a protecting group and the dotted lines mean that the remainder of the compound has the same structure as that of the compound of formula I, with a compound of formula III

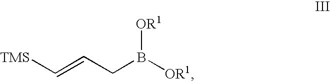

wherein R¹ is hydrogen C₁₋₈ alkyl or C₃₋₈ cycloalkyl and/or, when R¹ is hydrogen, trimer thereof;

or

a-ii) allylating a compound of formula II with a compound of formula IV

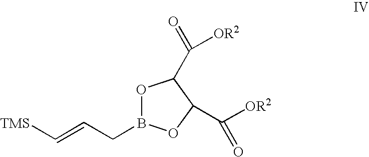

wherein R² is C₁₋₈ alkyl or C₃₋₈ cycloalkyl;

or

a-iii) allylating a compound of formula II with a compound of formula V

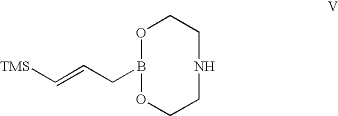

or

a-iv) allylating a compound of formula II with a compound of formula VI

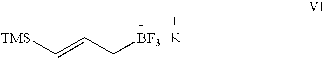

or

a-v) allylating a compound of formula II with a compound of formula VII

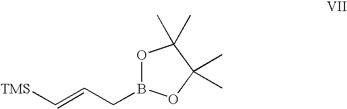

or

a-vi) allylating a compound of formula II with a reaction mixture obtained by a process comprising;

i) reacting allyltrimethylsilane with butyllithium to form trimethylsilylallyllithium;

ii) reacting trimethylsilylallyllithium with triisopropylborate or trimethylborate, and then conducting aqueous work up,

or

a-vii) allylating a compound of formula II with a reaction mixture obtained by reaction of the trimethylsilylallyllithium with diethylaluminum chloride,

or

a-viii) allylating a compound of formula II with a reaction mixture obtained by reaction of the trimethylsilylallyllithium with titanium tetraisopropoxide or titanium chlorotriisopropoxide,

to form a compound of formula XI;

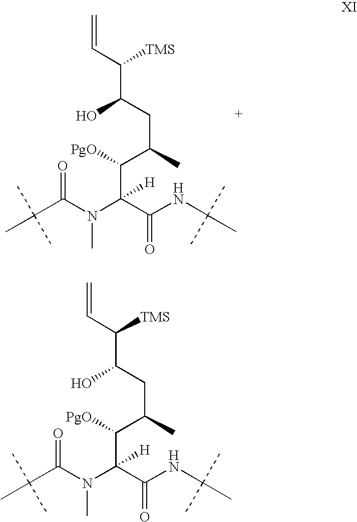

wherein Pg is as defined above;

and

b) converting the compound of formula XI to the cyclosporin A analog of formula I.

In a second aspect, this invention provides intermediates for the process mentioned above.

In a third aspect, this invention provides processes for the preparation of these intermediates.

Also, within the process as defined above [it will be referred to in the following under (i)], preferred are the following processes:

(ii) The process of (i), wherein step b) is conducted by

b-i) converting the compound of formula XI to a compound of formula XII

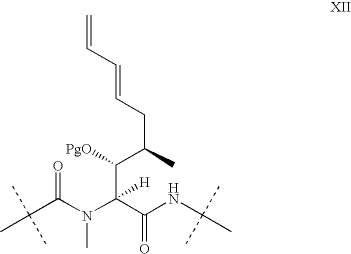

wherein Pg is as defined in (i),

under acidic conditions; and

b-ii) converting the PgO group of the compound of formula XII to a hydroxyl group.

(iii) The process of (i) or (ii), wherein Pg is acetyl group.

(iv) The process of (iii), wherein step a-i), a-ii) or a-vi) is conducted in the presence of tartrates.

(v) The process of (iii), wherein step a-i), a-ii) or a-vi) is conducted in dichloromethane or toluene.

(vi) The process of (iii), wherein step a-iii) is conducted in the presence of BF₃.Et₂O, formic acid, acetic acid or tartrate esters.

(vii) The process of (vi), wherein step a-iii) is conducted in water/dichloromethane or water/toluene.

(viii) The process of (vi), wherein step a-iii) and b-i) are conducted in dichloromethane or tetrahydrofuran and in the presence of BF₃.Et₂O.

(ix) The process of (vi), wherein step a-iii) is conducted in acetic acid and/or formic acid; or in a mixture of acetic acid and /or formic acid and one or two cosolvents selected from a group consisting of dichloromethane and tetrahydrofuran.

(x) The process of (ix), wherein step a-iii) is conducted in acetic acid and step b-i) is conducted by addition of formic acid to the reaction mixture.

(xi) The process of (ix), wherein step a-iii) and b-i) are conducted in formic acid or acetic acid/ formic acid.

(xii) The process of (i) or (ii), wherein step a-iv) is conducted in water/dichloromethane or water/toluene.

(xiii) The process of (iii), wherein step a-iv) and b-I) are conducted in dichlorometane, tetrahydrofuran or toluene in the presence of BF₃.Et₂O.

(xiv) The process of (iii), wherein step a-v) is conducted in the presence of BF₃.Et₂O.

(xv) The process of (xiv), wherein step a-v) and b-i) are conducted in dichloromethane, tetrahydrofuran or toluene and in the presence of BF₃.Et₂O.

(xvi) The process of (iii), wherein step a-v) is conducted in the presence of formic acid or acetic acid.

(xvii) The process of (xvi), wherein step a-v) and b-i) are conducted in formic acid or acetic acid/formic acid.

(xviii) The process of (xvii), wherein step a-v) and b-i) are conducted in a mixture of acetic acid/formic acid and co-solvent selected from dichloromethane, toluene, ethyl acetate and isopropyl acetate.

(xix) The process of (xviii), wherein co-solvent is isopropyl acetate.

(xx) The process of (xvi), wherein step a-v) is conducted in acetic acid and step b-i) is conducted by addition of formic acid to the reaction mixture.

(xxi) The process of (iii), wherein step a-vii) is conducted by allylating the compound of formula II with a reaction mixture prepared by reaction of the trimethylsilylallyllithium with diethylaluminum chloride.

(xxii) The process of (iii), wherein step a-viii) is conducted by allylating the compound of formula II with a reaction mixture prepared by reaction of trimethylsilylallyllithium with titanium tetraisopropoxide or titanium chlorotriisopropoxide.

### DETAILED DESCRIPTION OF THE INVENTION

The following terms used in the specification and claims have the meanings below:

“C_{a-b} alkyl” as used herein denotes straight chain or branched alkyl residues containing a to b carbon atoms. Therefore, for example, “C₁₋₈ alkyl” means straight chain or branched alkyl residues containing 1 to 8 carbon atoms, such as methyl, ethyl, propyl, isopropyl, butyl, isobutyl or tert.-butyl.

“C₃₋₈ cycloalkyl” refers to a saturated monovalent cyclic hydrocarbon radical of three to eight ring carbons e.g., cyclopropyl, cyclobutyl, cyclohexyl.

“Protecting group” refers to a grouping of atoms that when attached to a reactive group in a molecule masks, reduces or prevents that reactivity. Examples of protecting groups can be found in T. W. Green and P. G. Futs, Protective Groups in Organic Chemistry, (Wiley, 2^{nd} ed. 1991) and Harrison and Harrison et al., Compendium of Synthetic Organic Methods, Vols. 1-8 (John Wiley and Sons, 1971-1996). In particular, protecting groups in the present invention are carboxylic esters, i.e., an acyl group.

In the structural formulae presented herein a broken bond ( [symbol] ) denotes that the substituent is below the plane of the paper and a wedged bond ( [symbol] ) denotes that the substituent is above the plane of the paper.

The following abbreviations used in the specification and claims, otherwise specified, have the following significances:

MTBE methyl tert-butylether

THF tetrahydrofuran

DCM dichloromethane

DMSO dimethylsulfoxide

HMPA hexamethylphosphoramide

TMEDA tetramethylethylenediamine

TMS tetramethylsilane

TMS trimethylsilyl

Et ethyl

Me methyl

iPr isopropyl

Bu butyl

Ac acetyl

RT room temperature

HPLC high performance liquid chromatography

MS mass spectroscopy

TLC thin layer chromatography

NMR nuclear magnetic resonance spectroscopy

2D-COSY 2-dimensional correlated spectroscopy

2D-TOCSY 2-dimensional total correlation spectroscopy

HSQC Heteronuclear Single Quantum Coherence

Cryst. crystallization

Cpd compound

min. minute(s)

h hours

The starting materials and reagents used in the process of the present invention are either available from commercial suppliers such as Aldrich Chemical Co., (Milwaukee, Wis. USA), Bachem (Torrance, Calif. USA), Emka-Chemie, or Sigma (St. Louis, Mo. USA), Maybridge (Dist: Ryan Scientific, P.O. Box 6496, Columbia, S.C. 92960), Bionet Research Ltd., (Cornwall PL32 9QZ, UK), Menai Organics Ltd., (Gwynedd, N. Wales, UK), Butt Park Ltd., (Dist. Interchim, Montlucon Cedex, France), Fluka (CH-9471 Buchs, CH), Acros Organics (B-2440 Geel, BE) or are prepared by methods known to those skilled in the art following procedures set forth in references such as Fieser and Fieser's Reagents for Organic Synthesis, Volumes 1- 17 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 1992), and Larock's Comprehensive Organic Transformations (VCH Publishers Inc., 1989).

The starting materials and the intermediates of the reaction may be isolated and purified if desired using conventional techniques, including but not limited to filtration, distillation, crystallization, chromatography. Such materials may be characterized using conventional means, including physical constants and spectral data.

Compounds of formula I are prepared as illustrated in Scheme A. The allylmetal reagent also referred herein as allylating reagent is to be taken in a general sense and may comprise reagents where the metal part is based on boron although it is not per se a metal.

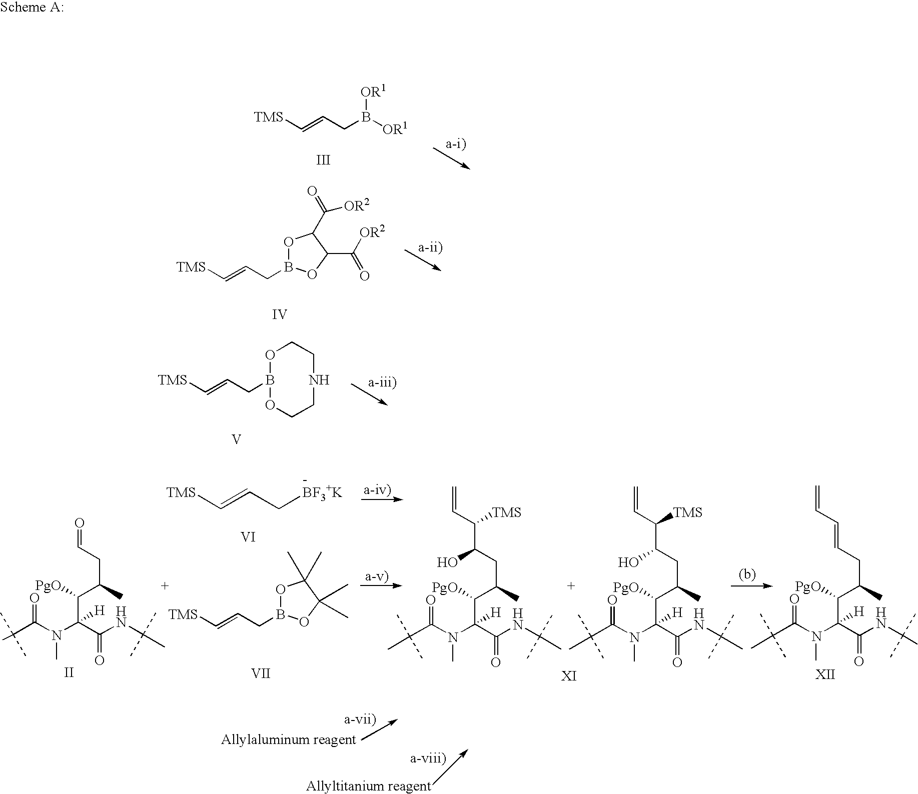

(Pg is a protecting group, the dotted lines mean that the remainder of the compound has the same structure as that of the compound of formula I, R¹ is hydrogen, C₁₋₈ alkyl or C₃₋₈ cycloalkyl and, when R¹ is hydrogen, a compound of formula III includes a trimer thereof, and R² is C₁₋₈ alkyl or C₃₋₈ cycloalkyl.)

In step a), protected cyclosporin A aldehyde of formula II is allylated by a γ-silylated allylmetal reagent of formula III, IV, V, VI, VII, VIII etc. to form a mixture of β-silylhomoallylic alcohol diastereomers of formula XI (For a general discussion about allylmetals and allylation of aldehydes see: W. R. Roush in “Allyl Organometallics”, Comprehensive Organic Synthesis, Pergammon Press, Vol 2, pp 1-53; Y. Yamamoto, N. Asao in “Selective Reactions Using Allylic Metals”, Chemical Reviews 1993, 93, p 2207-2293). The control of the relative anti or syn configuration of the β-silylalcohol fragment will depend on the allylmetal reagent and conditions used to perform the aldehyde allylation step (For a general discussion of γ-silyl substituted allylmetal reagents see: T. H. Chan in “Silylallyl Anions in Organic Synthesis: A Study in Regio- and Stereoselectivity”, Chemical Reviews 1995, 95, p1279-1292). This alcohol is often believed to form via a chair-like 6-membered ring transition state (also referred as Zimmerman-Traxler transition state) as shown in Scheme B.

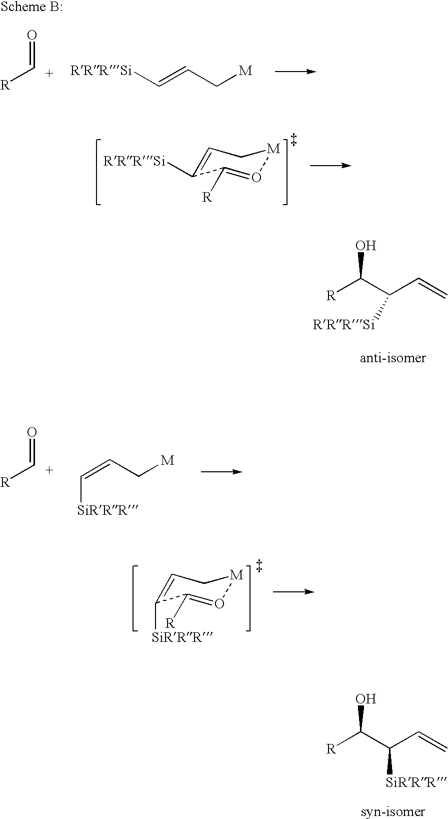

In such a transition state, the aldehyde side chain preferably adopts a pseudo equatorial position in order to minimize 1,3-diaxial steric interactions. The relative configuration of the β-silylalcohol fragment will therefore be determined by the configuration of C—C double bond of the allylmetal reagent.

Therefore, use of trans- or cis-γ-silylated allylmetals reagents should lead predominantly to the anti- or syn-β-silylalcohol isomer respectively. This holds in general, for example, for the allyl-boron, -titanium and -aluminum reagents.

Exception to this rule is found for example when a γ-silylated trialyklallylstannane reagent is added to aldehydes under Lewis acidic conditions, in that case the mechanism is different and the reaction provides mainly the syn β-silylalcohol isomer.

In step b), the β-silylalcohol of formula XI is converted to (E)-ISA247 of formula I.

Step b) can be carried out as illustrated in Scheme C.

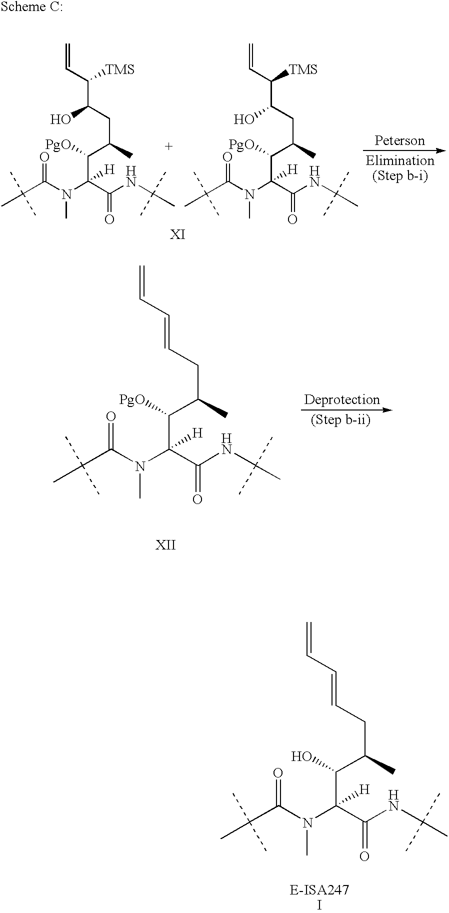

(Pg and the dotted lines have the meaning as defined above.)

In step b-i), the β-silylalcohol of formula XI undergoes a Peterson elimination (For a general discussion about Peterson eliminations, see: D. J. Ager in “The Peterson Reaction”, Synthesis 1984, p384-397 as well as references cited therein.) and the internal double bond is generated, i.e. the elimination of silanol from the β-silylalcohol moiety occurs.

In view of achieving a high degree of double bond isomeric purity, the success of the allylation-Peterson elimination sequence relies on the selective introduction of a relative anti or syn configuration of the β-silylalcohol moiety.

Indeed the Peterson elimination is known to be stereospecific. Anti isomers will provide one isomeric double bond when the syn isomers will produce the other double bond isomer under the same conditions as illustrated in Scheme D.

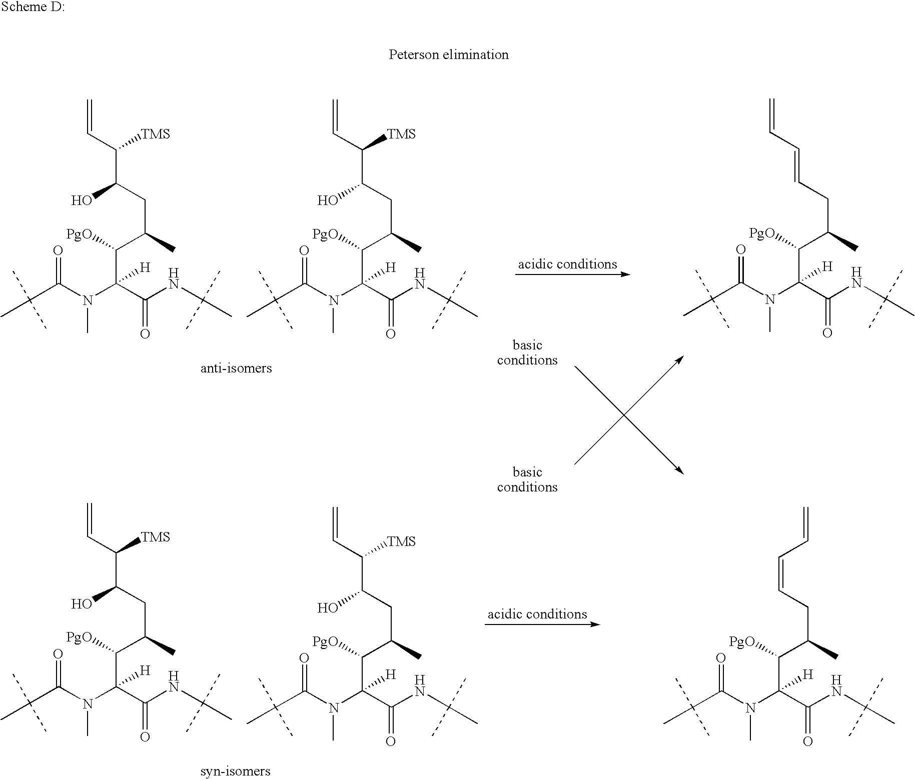

(Pg and the dotted lines have the meaning as defined above.)

Anti isomers should give the trans double bond under acidic Peterson elimination conditions whereas syn isomers would provide the cis double bond. The reaction proceeds via a mechanism where the hydroxyl and the silyl groups are in an anti conformation prior to elimination.

The situation is opposite when the Peterson elimination is performed under basic conditions, in that case the reaction proceeds via a mechanism where the deprotonated hydroxyl and the silyl group are in a syn conformation prior to elimination.

Therefore, in principle, one could reach either double bond isomer by controlling the formation of either the syn or anti relative configuration of the β-silylalcohol moiety or by using either acid or basic Peterson elimination conditions.

In the present invention, trans-γ-silylated allylmetals reagents are used for allylation of protected cyclosporin A aldehyde of formula II to form a mixture of anti-β-silylalcohol diastereomers of formula XI. Therefore, a Peterson elimination is performed under acidic condition to form a trans double bond.

Typical acids for the acid-promoted reaction may include sulfuric acid, formic acid, hydrochloric acid, methanesulfonic acid tetrafluoroboric acid, perchloric acid, trifluoroacetic acid and various Lewis acids. Preferred acids are sulfuric acid, formic acid, methanesulfonic acid and BF₃.Et₂O, especially sulfuric acid, formic acid and BF₃.Et₂O. This step can be conducted at a reaction temperature from −70° C. to 50° C. Preferred temperature range is 0° C. to 50° C., more preferably 20° C. to 40° C. for formic acid. sulfuric and methanesulfonic acid. Preferred temperature range is −80° C. to 50° C., preferably −80° C. to 25° C., especially −80° C. to 0° C. for BF₃.Et₂O.

E-acetyl-ISA247 can be purified by crystallization in MTBE (for example via solvent exchange from dichloromethane to MTBE) or in MeOH/water mixtures.

In step b-ii), the protecting group is removed, returning the functional group on that carbon to an alcohol. The conditions and reagents to be employed depend on the protecting group used, which are known to those skilled in the art. One such protecting group employed in the present invention is an acyl group (R′C(O)—; wherein R′ is a linear saturated monovalent hydrocarbon radical of one to six carbon atoms or a branched saturated monovalent hydrocarbon radical of three to six carbon atoms), such as acetyl, propionyl, butyryl, isobutyryl, valeryl can preferably be used as a protecting group. When the protecting group is an acetyl group, it can be removed, for example, by the treatment with K₂CO₃ in methanol and water. Under these conditions, the isomeric purity of the diene fragment is preserved. Therefore the double bond isomeric purity of E-ISA247 reflects the double bond isomeric purity of E-acetyl-ISA247. Bases other than potassium carbonate that may be used to remove the protecting group include sodium hydroxide, sodium carbonate, sodium alkoxide and potassium alkoxide.

### Synthesis of (E)-ISA247 by Allylboron Reagents

Allylation by γ-Silylated Allylboron Reagent of Formula III or IV (Step a-i) and a-ii))

In general, excess of the reagent of formula III or IV is needed to complete the allylation of acetylcyclosporin A aldehyde (II′) within an acceptable timeframe. Higher conversion and rates are achieved by using an activating agent such as a tartrate ester and/or dichloromethane as (co)-solvent. In accordance to the general behavior of boronic acids, the reagent of formula IIIa can potentially exist in the form of cyclic trimer (boroxine) or oligomers (For an example of such behavior of a boronic acid, see: K. Ishihara, H. Kurihara, M. Matsumoto and H. Yamamoto in “Design of Bronsted Acid-Assisted Chiral Lewis Acid (BLA) Catalysts for Highly Enantioselective Diels-Alder Reactions”, Journal of the American Chemical Society 1998, 120, p6920-6930.). When triisopropylborate is used for the preparation of the solution of the crude reagent of formula IIIa, reagent of formula IIIa can also contain diisopropyl boronate ester (TMS—CH═CH—CH₂—B (OiPr)₂), from isopropanol generated from B(OiPr)₃) and mixed derivatives such as TMS—CH═CH—CH₂—B(OH)(OiPr). This solution can be used as an allylation reagent without purification.

Alternatively, a solution of reagent of formula IIIa can be generated by hydrolysis of complex of formula V in organic solvent/water mixture such as a dichloromethane/water, toluene/water, ethyl acetate/water, THF/water, chloroform/water mixture, preferably a dichloromethane/water mixture, preferably in the presence of an acid such as sulfuric acid, hydrochloric acid, acetic acid, preferably acetic acid. Allylation of acetylcyclosporin A aldehyde with a dichloromethane solution of reagent of formula IIIa prepared as just described can reach high conversions using as low as 2 equivalents of the reagent. In this case, isopropyl derivatives are of course absent.

Without tartrate activation

Toluene can be used as solvent for these reactions, however, marked solvent effects have been observed in these reactions. The allylation is best performed in polar non-coordinating solvents, preferably dichloromethane.

When tartrate additive is omitted, allylation is preferably performed in dichloromethane using a concentrated solution of the crude boronic acid (>10%, preferably ca 50% concentration).

Preferred reagent of formula III wherein R¹ is hydrogen, C₁₋₈ alkyl or C₃₋₈ cycloalkyl and/or, when R¹ is hydrogen, a trimer thereof are those wherein R¹ is hydrogen, methyl, ethyl, propyl, isopropyl, butyl or benzyl, more preferably R¹ is hydrogen, methyl, ethyl, propyl, isopropyl or butyl, further preferably hydrogen, methyl, ethyl, propyl or butyl, especially preferably hydrogen. Allylation is performed in organic solvent such as ethyl acetate, THF, toluene, chloroform or dichloromethane, preferably in ethyl acetate, toluene or dichloromethane, more preferably in toluene or dichloromethane, especially in dichloromethane.

For example, the synthesis of (E)-ISA247 by the reagent of formula III can be carried out as illustrated in Scheme E.

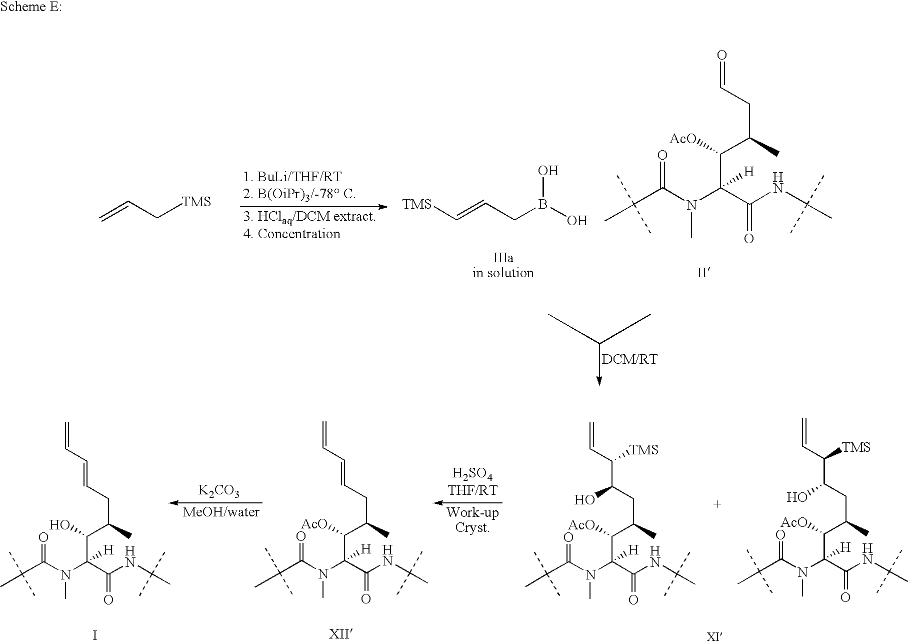

(R¹ and the dotted lines have the meaning as defined above.)

The allylation of acetyl-cylcosporine A aldehyde (II′) in dichloromethane with 10 equiv. of a ca 50% solution of boronic acid reaches over 95% conversion within 60 min at RT and yields a mixture of anti β-trimethylsilylalcohol diastereomers (XI′). The Peterson elimination can be performed after aqueous work-up on the crude allylation product at 0° C. to RT in THF with sulfuric acid. Alternatively, the Peterson elimination can take place directly on the allylation reaction mixture by addition of THF and sulfuric acid. Aqueous work-up and crystallization yields the (E)-acetyl-ISA247 (XII′). Hydrolysis of the (E)-acetyl-ISA247(XII′) provides (E)-ISA247 (I).

With Tartrate Activation

As shown in Scheme F, the addition of a tartrate ester, such as, for example, L-(+)-dimethyltartrate in the presence of a drying agent, activates the boronic acid of formula IIIa by generating in situ the corresponding boronate ester, a reagent class known in the literature to exhibit very high allylation reactivity. The reaction then proceeds partially or mainly through the generated boronate ester increasing the rate of the allylation.

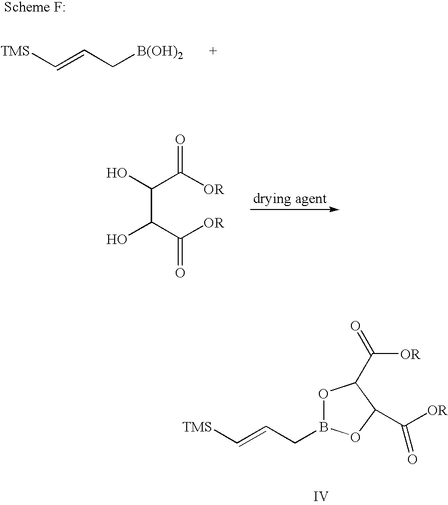

(R is C₁₋₈ alkyl, preferably C₁₋₆ alkyl, more preferably methyl, ethyl or isopropyl, especially methyl.)

Allylation with reagent of formula IV is performed in organic solvent such as ethyl acetate, THF, toluene, chloroform or dichloromethane, preferably in ethyl acetate, toluene or dichloromethane, more preferably in toluene or dichloromethane, especially in dichloromethane.

For example, the synthesis of (E)-ISA247 by the reagent of formula IV is performed as illustrated in Scheme G.

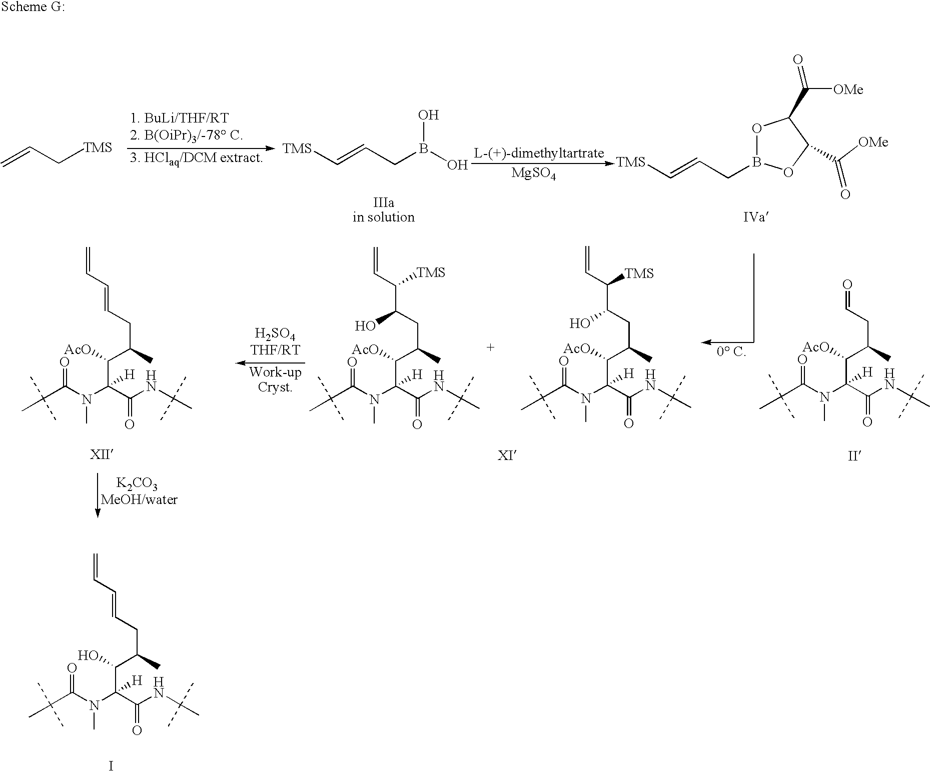

(The dotted lines have the meaning as defined above.)

The generation of the reagent of formula IVa′ by mixing a solution of boronic acid of formula IIIa with L-(+)-dimethyltartrate, in the presence of a drying agent such as molecular sieves or magnesium sulfate, preferably magnesium sulfate , is evidenced by ¹¹B and ¹H NMR analyses.

Allylation of acetylcyclosporin A aldehyde (II′) with a boronic acid reagent in situ activated by addition of L-(+)-dimethyltartrate at a temperature of 0° C. to RT, preferably at 0° C., give a mixture of anti β-silylalcohol diastereomers (XI′). Aqueous work-up followed by the Peterson elimination in THF with sulfuric acid provides, after work-up and crystallization, (E)-acetyl-ISA247 (XII′). Hydrolysis of the acetyl protecting group yields (E)-ISA247 (I).

Care should be taken that reaction involving the use of crude boronic acid solution with or without tart rate activation should be performed at neutral or acidic pH (between 3 and 7, preferably between 5 and 6). Indeed when the pH is over 7, substantial amount of a side-product identified as the vinylsilane of formula XV are formed. A test reaction (performed without tartrate activation) where Et₃N amine was added to reach a pH of 9-10 led to the almost exclusive formation of the vinylsilane product XV (as evidenced by MS, ¹H NMR, COSY, TOCSY and HSQC NMR experiments). Such an effect was totally unexpected.

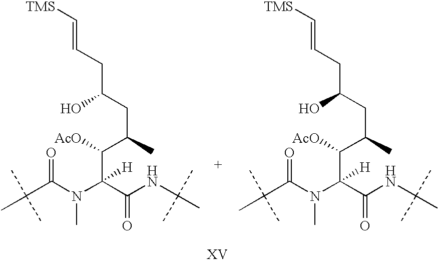

(The dotted lines have the meaning as defined above.)

Allylation by γ-Silylated Allylboron Reagent of Formula V (Step a-iii))

Without activation, the diethanolamine complex of formula V does not react at RT with acetylcyclosporin A aldehyde in non protic solvents like dichloromethane or THF. However, the complex of formula V represent a stable source of the corresponding boronic acid.

When treated in a water/organic solvent, (such as ethyl acetate, THF, dichloromethane or toluene, preferably ethyl acetate, dichloromethane or toluene, more preferably dichloromethane) mixture preferably in the presence of acid such as sulfuric acid, hydrochloric acid or acetic acid, preferably acetic acid, the diethanolamine complex V is hydrolyzed and liberates the reactive boronic acid as shown, for example, in Scheme H, which can then reacts with the acetylcyclosporin A aldehyde (II′), preferably at RT.

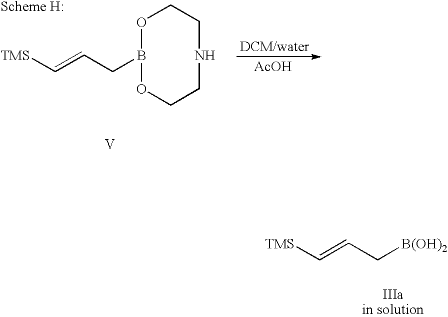

Allylation of acetylcyclosporin A aldehyde (II′) under such conditions provides a mixture of anti β-trimethylsilylalcohol diasteromers (XI′). After the water phase is discarded, solvent is exchanged to THF, and sulfuric acid is added to perform the Peterson elimination. Aqueous work-up and crystallization provides (E)-acetyl-ISA247 (XII′). Subsequent hydrolysis yields (E)-ISA247 (I). Alternatively, isolation of the crude anti β-trimethylsilylalcohol diasteromers after aqueous work-up, followed by Peterson elimination under standard conditions (concentrated sulfuric acid in THF) furnishes E-acetyl-ISA247.

For example, the synthesis of (E)-ISA247 by the complex of formula V can be performed as illustrated in Scheme I.

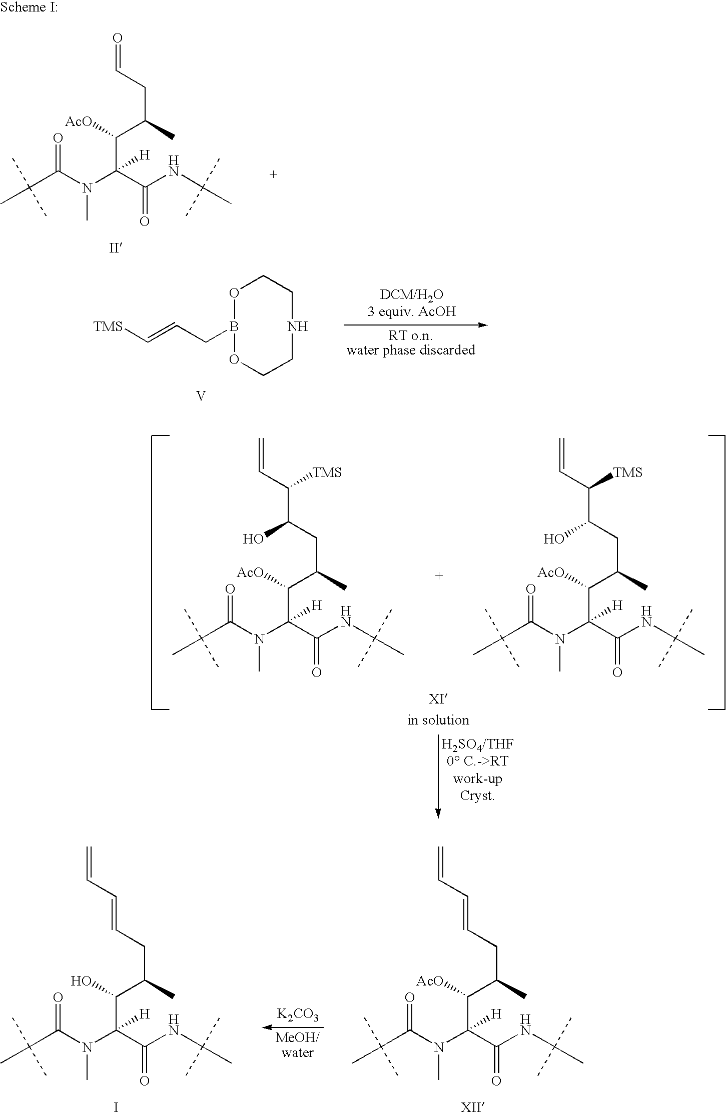

(The dotted lines have the meaning as defined above; o.n. designates overnight.)

Allylmetalation of acetylcyclosporin A aldehyde (II′) can also take place under non-aqueous conditions directly with complex of formula V. Indeed, protic solvents such as carboxylic acids are particularly effective. Solvent mixture could be acetic acid and/or formic acid or a combination of acetic acid and/or formic acid and a co-solvent such as dichloromethane and THF. The allylation is best performed in acetic acid between RT and 35° C. This provides a mixture of anti β-silylalcohol diastereomers (XI′). These intermediates could of course be isolated but the Peterson elimination can be conducted in one pot by addition to the reaction mixture of an acid such as formic acid, sulfuric acid or methanesulfonic acid, preferably formic acid. Aqueous work-up and crystallization yields (E)-acetyl-ISA247 (XII′). Subsequent hydrolysis furnishes (E)-ISA247 (I).

When formic acid is present in sufficient amounts in the solvent mixture used for the allylation, the Peterson elimination can take place in one-pot leading directly to (E)-acetyl-ISA247.

Another alternative consists in performing the addition of complex of formula V to acetylcyclosporin A aldehyde (II′) in the presence of a Lewis acid such as BF₃.Et₂O. For example, the reaction with BF₃.Et₂O can be performed in a solvent such as dichloromethane or THF at a temperature ranging from −40° C. to RT. Under these conditions, the allylation can directly be followed by the Peterson elimination, yielding the expected (E)-acetyl-ISA247 (XII′).

Allylation by γ-Silylated Allylboron Reagent of Formula VI (Step a-iv))

Reacting the allyltrifluoroborate VI and acetylcyclosporin A aldehyde (II′) in a biphasic water/organic solvent, preferably water/dichloromethane mixture or water/toluene mixture, more preferably water/dichloromethane mixture at RT provides a mixture of anti β-trimethylsilylalcohol diastereomers (XI′). After the water phase is discarded, the Peterson elimination is performed by addition of THF and sulfuric acid at a temperature of 0° C. to RT providing (E)-acetyl-ISA247 (XII′). Peterson elimination can also be performed under standard conditions (sulfuric acid in THF) after isolation of the anti β-trimethylsilylalcohol diastereomers to give E-acetyl-ISA247.

The allylation can also be promoted by a Lewis acid. In that case, the allylation and the Peterson elimination can be combined in a one-pot process. For example, addition of excess BF₃.Et₂O to a suspension of allyltrifluoroborate VI (2 equiv.) in a solution of acetyl-cyclosporin A aldehyde (XII′) in dichloromethane at −70° C. provides after 60 min. reaction and aqueous work-up, (E)-acetyl-ISA247 (I). Solvents for the reaction are organic solvent such as dichloromethane, THF or toluene, preferably dichloromethane.

Allylation by γ-Silylated Allylboron Reagent of Formula VII (Step a-v))

Allylation of aldehydes with this reagent is known from the literature to proceed slowly (1 to several days at RT). Accordingly, allylation of acetylcyclosporin A aldehyde (II′) with excess of reagent of formula VII (5-10 equivalents) in solvents such as THF, dichloromethane, toluene, DMF and DMSO proceeds slowly at RT.

Heating, use of large excess of reagent or high concentration could increase the rate of acetyl cyclosporin A aldehyde allylation, however, a better alternative was found by a proper choice of solvent.

Carboxylic acids such as formic acid or acetic acid were found to dramatically enhance the rate of allylation. For instance, when performed in acetic acid, allylation of acetyl cyclosporin A aldehyde (II′) can reach conversion of over 95% within 5 hours at RT with 2 equivalents of reagent of formula VI, providing the β-silylalcohols (XI′). Further addition of formic acid promotes the Peterson elimination. (E)-acetyl-ISA247 (XII′) is obtained after extractive work-up ascertaining the relative anti stereochemistry of the intermediate β-silylalcohols. Peterson elimination can also be performed under standard conditions (sulfuric acid in THF) after isolation of the anti β-trimethylsilylalcohol diastereomers to give (E)-acetyl-ISA247.

Formic acid or preferably a combination of acetic acid and formic acid (such as ca 1:1 v/v) as is also an effective solvent. In that case, the allylmetalation and the following Peterson elimination can take place in one-pot. In a combination of acetic acid and formic acid (ca 1:1 v/v) the allylation of acetylcyclosporin A aldehyde and the following Peterson elimination reach over 90% conversion within 60 min. at RT with 1.5 equivalent of reagent. Aqueous extractive work-up and crystallization furnishes (E)-acetyl-ISA247 (XII′).

Mixture of acetic acid, formic acid and a suitable co-solvent can also be used. Dichloromethane, toluene, ethyl acetate or isopropyl acetate, preferably isopropyl acetate could be used as co-solvent. Decrease in reactivity can be observed when using a co-solvent but this could be compensated by increasing the reaction temperature.

Hydrolysis of the acetate protecting group with K₂CO₃ in aqueous methanol furnishes (E)-ISA247 (I).

For example, the synthesis of (E)-ISA247 by the reagent of formula VI can be performed as illustrated in Scheme J.

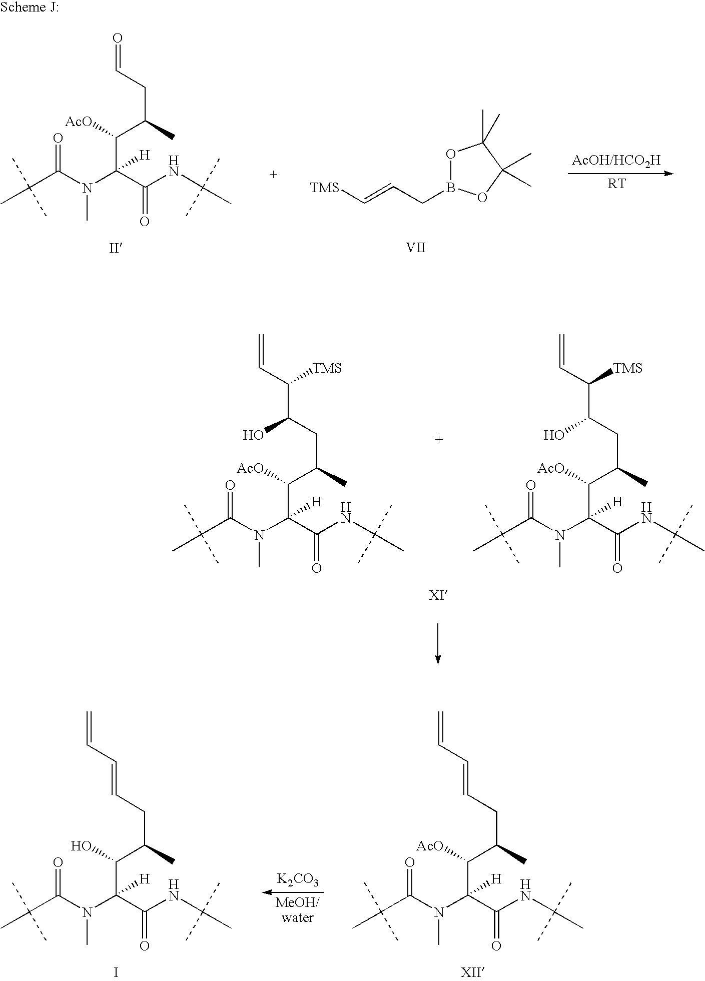

The origin of the increased activity of the reagent of formula VII, when used in carboxylic acid solution like acetic acid and formic acid, could come from the high polarity and the low complexing ability of these solvents. Another effect could be found in the ability of these solvent to provide acidic catalysis of the allylation by activation of the carbonyl group of the aldehyde through protonation. By protonation of the boronate oxygens, these acids may enhance the elctrophilicity of boron.

The addition of reagent of formula VII to acetyl cyclosporin A aldehyde (II′) can be promoted by a Lewis acid such as BF₃.Et₂O at a temperature of −70° C. to 0° C. in toluene, THF or dichloromethane, preferably toluene or dichloromethane, preferably dichloromethane. Under the allylation reaction, the Peterson elimination also occurs and (E)-acetyl-ISA247 (XII′) can be obtained after extractive aqueous work-up.

Synthesis of (E)-ISA247 by Allyltitanium Reagents

Reaction of acetylcyclosporine A aldehyde with a γ-trimethylsilylallyltitanium reagent prepared from trimethylsilylallyllithium and titanium dichlorodiisopropoxide, titanium tetraisopropoxide or titanium chlorotriisopropoxide, preferably titanium tetraisopropoxide or titanium chlorotriisopropoxide performed in THF, at a temperature of −80° C. to 0° C., preferably −80° C. to −30° C., more preferably −80° C. to −50° C., especially −80° C. to −60° C., furnishes, after aqueous work-up, a mixture of diastreomeric anti β-silylalcohols XI′. Peterson elimination under the standard conditions (H₂SO₄ in THF) furnishes E-acetyl-ISA247 (XII′).

Synthesis of (E)-ISA247 by Allylaluminum Reagents

Reaction of acetylcyclosporine A aldehyde with a γ-trimethylsilylallylaluminum reagent prepared from trimethylsilylallyllithium and ethylaluminum dichloride or diethylaluminum chloride, preferably diethylaluminum chloride, performed in THF, at a temperature of −80° C. to 0° C., preferably −80° C. to −30° C., more preferably −80° C. to −50° C., especially −80° C. to −60° C. furnishes after aqueous work-up a mixture of diastreomeric anti β-silylalcohols XI′. Peterson elimination under the standard conditions (H₂SO₄ in THF) furnishes E-acetyl-ISA247 (XII′).

Preparation of the γ-Silylated Allylmetal Reagents

The γ-silylated allylmetal reagents required for the allylmetalation step are best generated from the corresponding allylsilanes via deprotonation, trapping with an adequate metal reagent and optionally by further complexation of the metal rest by a suitable ligand. The resulting reagents can, depending on their stability and the process, be used in situ, i.e. directly in solution, or be isolated and stored.

Although other silyl-substituted allylsilanes could obviously have been used, the trimethylsilyl derivative leads to minimum amount of waste. Indeed, the silyl fragment is lost upon Peterson elimination.

Many conditions for the deprotonation of allylsilanes have been published in the literature and usually make use of n-butyllithium (or the sec- and tert-isomers) in an organic solvent (generally THF) in combination or not with a co-solvent or co-reagent such as HMPA, TMEDA or potassium t-butoxide at temperatures ranging from −100° C. to RT. (see for example: T. H. Chan in “Silylallyl Anions in Organic Synthesis: A Study in Regio- and Stereoselectivity”, Chemical Reviews 1995, 95, p1279-1292; Kohei Tamao, Eiji Nakajo, and Yoshihiko Ito in “Silafunctional compounds in organic synthesis. 33. Metalated allylaminosilane: a new, practical reagent for the stereoselective alpha.-hydroxyallylation of aldehydes to erythro-1,2-diol skeletons”, Journal of Organic Chemistry 1987, 52, pp 957-958; E. Ehlinger and P. Magnus in “Silicon in Synthesis. 10. The (Trimethylsilyl)allyl Anion: A β-Acyl Anion Equivalent for the Conversion of Aldehydes and Ketones into γ-Lactones”, Journal of the American Chemical Society 1980, 102, pp 5004-5011; M. Schlosser, L.Franzini in “The Regioselectivity of 1,3-Disubstituted Allylmetal Species Towards Electrophiles: 1-(Trimethylsilyl)alk-2-enylpotassium Compounds”, Synthesis 1998, pp 707-709; E. Schaumann and A. Kirschning in “Ring-opening of oxiranes by silyl-substituted allyl anions. A regiochemical chameleon”, Tetrahedron Letters 1988, 29, pp 4281-4284).

As illustrated in Scheme K, the allyltrimethylsilane is deprotonated by n-butyllithium in THF at a temperature ranging from 0° C. to 35° C., preferably bewteen 0° C. and 25° C. for 30 min. up to 3 hours. This generates a trimethylsilylallyllithium intermediate. This intermediate most probably exists in solution as a π-allyl complex of lithium in a trans configuration (T. H. Chan in “Silylallyl Anions in Organic Synthesis: A Study in Regio- and Stereoselectivity”, Chemical Reviews 1995, 95, p1279-1292; M. Schlosser, O. Desponds, R. Lehmann, E. Moret and G. Rauschschwalbe in “Polar Allyl Typer Organometallics as Key Intermediates in Regio- and Stereocontrolled Reactions: Conformational Mobilities and Preferences”, Tetrahedron 1993, 49, p10175-10203). This anion is then trapped (transmetalated) by an electrophilic metal source usually at a temperature of −80° C. to −60° C., generating the corresponding trans-allylmetal reagent. Depending on the metal rest, these reagents are reacted in situ with the aldehyde or can be isolated for later use. It can also be transformed into another complex by addition of a proper reagent in order to activate the reagent for the allylation or to allow their isolation.

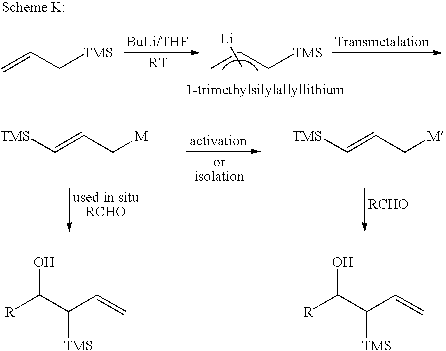

(M and M′ are a metallic fragment comprising the metal and its ligands.)

### Preparation of Allylboron Reagents

γ-Silylated Allylboron Reagent of Formula IIIa

A solution of crude boronic acid of formula IIIa is obtained after deprotonation of allyltrimethylsilane, trapping with an electrophilic boron reagent and aqueous work-up. The deprotonation of allyltrimethylsilane is performed in THF with butyllithium, between 0° C. and 35° C., preferably between 0° C. and 25° C. for 30 min. to 3 hours. The electrophilic boron reagent is a trialkylborate such as triisopropyl borate or trimethyl borate, preferably triisopropyl borate. The trapping of the trimethylsilylallyllithium intermediate with triispropyl borate is performed between −80° C. and −20° C., preferably below −60° C. for 30 min. to 2 hours. The trapping of the trimethylsilylallyllithium intermediate with trimethyl borate is performed between −80° C. and −60° C. for 30 min. to 2 hours.

It is known that allylboronic acids are unstable, therefore, the crude boronic acid is kept in solution (P. G. M. Wuts and Y.-W. Jung in “The Addition of γ-(Trimethylsilyl)allylboronates to Imines”, Journal of Organic Chemistry 1991, 56, p365-372(see comment in the example for the preparation of compound 9); W. R. Roush, K. Ando, D. B. Powers, A. D. Palkowitz and R. L. Halterman in “Asymmetric Synthesis Using Diisopropyl Tartrate Modified (E)- and (Z)-Crotylboronates; Preparation of the Chiral Crotylboronates and Reactions with Achiral Aldehydes”, Journal of the American Chemical Society 1990, 112, 6339 (see reference 17)). Concentrated solutions (>10% concentration, for example ca 50% concentration) are not stable at RT and decompose. They should be rapidly used. If needed they should be stored at 5° C. maximum. In accordance to the general behaviour of boronic acids, the boronic acid of formula IIIa might in principle also exists in the form of a cyclic trimer (also called a boroxine) or oligomers. Since isopropanol coming from triisopropyl borate can also present (depending on the process), (mixed) isopropyl boron esters (TMS—CH═CH—CH₂—B(OH)(OiPr) and/or TMS—CH═CH—CH₂—B (OiPr)₂) could also be present. Indeed, ¹H NMR and ¹¹B NMR analyses of concentrated solutions of boron reagent of formula IIIa (ca 50%) diluted with an equal volume of CD₂Cl₂ shows the presence of several boronate species, although reverse phase HPLC analysis shows mainly the boronic acid (under the HPLC conditions, the oligomers, dimers and trimers should be hydrolyzed and converted to the boronic acid).

Alternatively, a solution of reagent of formula IIIa can be prepared by hydrolysis of complex of formula V in an organic solvent/water mixture such as a dichloromethane/water, toluene/water, ethyl acetate/water, THF/water, chloroform/water mixture, preferably a dichloromethane/water mixture preferably in the presence of an acid such as sulfuric acid, hydrochloric acid, acetic acid, preferably acetic acid.

Preparation of the γ-silylated allylmetal reagent of formula IIIa is, for example, performed as illustrated in Scheme L.

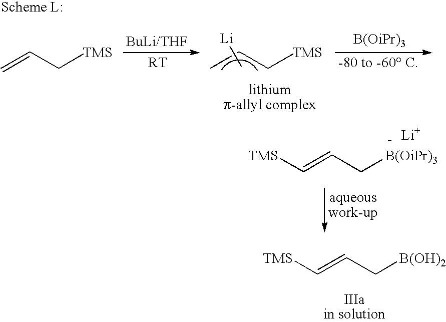

γ-Silylated Allylboron Reagent of Formula IV

Boronate reagent of formula IV where R is C₁₋₈ alkyl, preferably C₁₋₆ alkyl, more preferably methyl, ethyl or isopropyl, especially methyl is prepared by treating boron reagent of formula IIIa with the required tartrate ester in the presence of a drying agent such molecular sieves or magnesium sulfate, preferably magnesium sulfate.

Reagent of formula IVa′

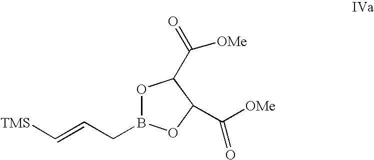

is prepared by mixing a solution of boronic acid of formula IIIa with L-(+)-dimethyltartrate, in the presence of a drying agent such molecular sieves or magnesium sulfate, preferably magnesium sulfate, as evidenced by ¹¹B and ¹H NMR analyses.

γ-Silylated Allylboron Reagent of Formula III

This reagent can be prepared by treating boron reagent of formula IIIa with the required alcohol in the presence of a drying agent such molecular sieves or magnesium sulfate, preferably magnesium sulfate. Preferred boronate reagent of formula IV wherein R¹ is C₁₋₈ alkyl or C₃₋₈ cycloalkyl are those wherein R¹ is methyl, ethyl, propyl, isopropyl, butyl or benzyl, more preferably R¹ is methyl, ethyl, propyl, isopropyl or butyl, further preferably R¹ is methyl, ethyl, propyl or butyl, especially preferably R¹ is methyl.

γ-Silylated Allylboron Reagent of Formula V

To a solution of crude boronic acid of formula IIIa, preparation of which was described above, is added diethanolamine. Solvent exchange to heptane and crystallization provides the diethanolamine complex of formula V as a solid. A special feature of this reagent is the presence of an interaction between the nitrogen lone pair of the diethanolamine fragment and the boron atom (i.e. complexation of the nitrogen atom by the boron atom) as evidence by ¹¹B NMR of the complex (δ=11 ppm relative to BF₃.Et₂O, external reference).

Preparation of the γ-silylated allylmetal reagent of formula V is, for example, performed as illustrated in Scheme M:

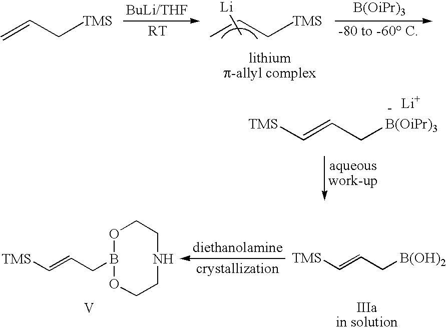

γ-Silylated Allylboron Reagent of Formula VI

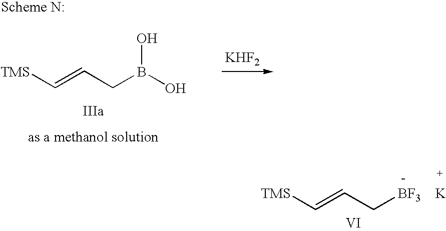

In general, allyltrifluoroborate potassium salts are prepared by treating the corresponding boronic acid with 3 equivalents of KHF₂ in a water/methanol solvent mixture (see for example: R. A. Batey in “Diastereoselective Allylation and Crotylation Reactions of Aldehydes with Potassium Allyl- and Crotyltrifluoroborates under Lewis acid Catalysis”, Synthesis 2000, pp 990-998). However, direct application of these procedures to the preparation of trifluoroborate of formula VI lead to the formation of substantial amounts of allyltrimethysilane via protodeborylation due to the acidic pH of the reaction mixture.

Modification was made in order to avoid this side-reaction. Thus, as illustrated in Scheme O, a methanol solution of boronic acid is treated with 2 equivalents of KHF₂ as fluoride source, at RT. The suspension is stirred at RT for 60 min. The residual organic salts are removed by filtration. The methanolic solution of trifluoroborate salt VI is concentrated under reduced pressure and the product is crystallized at 0-5° C. The trifluoroborate salt VI is isolated by filtration and dried under vacuum.

The required boronic acid solution is prepared by hydrolyzing the diethanolamine complex of formula V in a water/dichloromethane mixture in the presence of an acid such as acetic acid. The aqueous phase is discarded and the solvent is exchanged from dichloromethane to methanol.

Alternatively, the diethanolamine complex V can be used directly as starting material. The trifluoroborate salts VI can be prepared, however, crystallization does not occur.

γ-Silylated Allylboron Reagent of Formula VII

To a solution of crude boronic acid of formula IIIa, preparation of which was described above, is added pinacol. The reaction mixture is stirred at RT and then concentrated under reduced pressure. The pinacol complex VII can then be distilled under low pressure or used directly in the allylmetalation step. The preparation of the pinacol complex of formula VII is, for example, performed as illustrated in Scheme O.

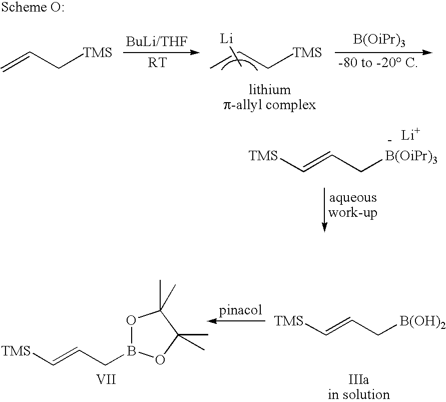

Alternatively, the trapping of the 1-trimethylsilylallyl lithium can be performed with 2-isopropoxy-4,4,5,5-tetramethyl-1,3,2-dioxaborolane, leading directly after aqueous work-up to the pinacol boronate of formula VII.

Alternatively, the reagent can be prepared by deprotonation of allyltrimethylsilane at room temperature with butyllithium, quench of the allyllithium intermediate with tri-ispropylborate (between −80° C. to −20° C., preferably between −80° C. and −30° C.), addition of pinacol and then aqueous work-up.

### Preparation of the Allyltitanium Reagents

The (trimethylsilyl)allyltitanium reagents are prepared in situ via deprotonation of allyltrimethylsilane to form trimethylsilylallyllithium, as described above, and reaction of this intermediate with titanium dichlorodiisopropoxide, titanium tetraisopropoxide or titanium chlorotriisopropoxide, preferably titanium tetraisopropoxide or titanium chlorotriisopropoxide at a temperature of −80° C. to 0° C., preferably −80° C. to −30° C., more preferably −80° C. to −50° C., especially −80° C. to −60° C. The resulting titanium reagents are used in situ for the allylation of protected cyclosporin A aldehydes. Putative structures for theses reagents are presented below:

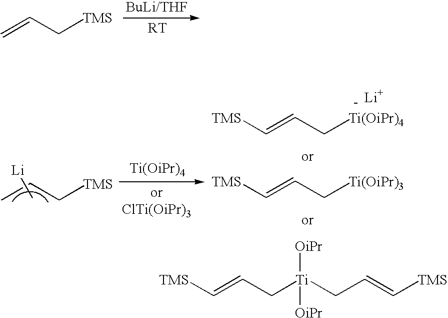

### Preparation of the Allylaluminum Reagents

The (trimethylsilyl)allylaluminum reagents are prepared in situ via deprotonation of allyltrimethylsilane to form trimethylsilylallyllithium, as described above, and reaction of this intermediate with a dialkylaluminum chloride such as diethylaluminum chloride or with an alkylaluminum dichloride such as ethylaluminum dichloride, preferably with diethylaluminum chloride, at a temperature of −80° C. to 0° C., preferably −80° C. to −30° C., more preferably −80° C. to −50° C., especially −80° C. to −60° C. The resulting aluminum reagents are used in situ for the allylation of protected cyclosporin A aldehydes.

A putative structure for one of these reagents is presented below:

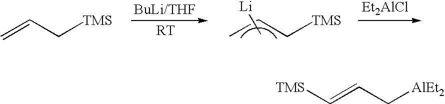

### Preparation of Protected Cyclosporin A Aldehyde

Protected cyclosporin A aldehyde of formula II can be prepared as illustrated in Scheme P.

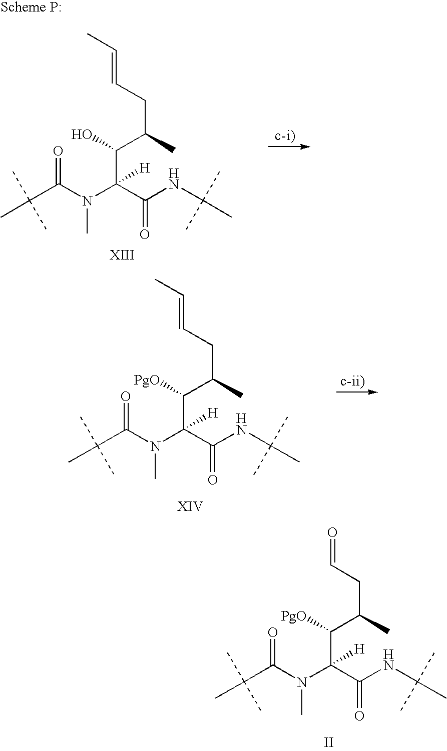

(The dotted lines have the meaning as defined above.)

In step c-i), a protecting group is introduced in cyclosporin A of formula XIII, to protect hydroxyl group at the β-position of the side chain of the 1-amino acid residue. Protecting groups are well known in organic synthesis, and have been discussed by J. R. Hanson in Chapter 2, “The Protection of Alcohols,” of the publication Protecting Groups in Organic Synthesis (Sheffield Academic Press, Sheffield, England, 1999), pp. 24-25. Hanson teaches how to protect hydroxyl groups by converting them to either esters or ethers. Acetate esters are perhaps the most frequently used type of chemistry for protecting hydroxyl groups. There are a wide range of conditions that may be used to introduce the acetate group. These reagents and solvents include acetic anhydride and pyridine; acetic anhydride, pyridine and dimethylaminopyridine (DMAP); acetic anhydride and sodium acetate; acetic anhydride and toluene-p-sulphonic acid, acetyl chloride, pyridine and DMAP; and ketene. DMAP is a useful acylation catalyst because of the formation of a highly reactive N-acylpyridium salt from the anhydride.

For example, the β-alcohol of cyclosporin A is protected as an acetate by reacting cyclosporin A (XIII) with acetyl chloride, ethyl acetate, or combinations thereof, forming the compound, acetyl cyclosporin A. In another example, the β-alcohol undergoes a nucleophilic addition to acetic anhydride, forming acetyl cyclosporin A and acetic acid. These reactions may be carried out in the presence of dimethylaminopyridine (DMAP) where an excess of acetic anhydride acts as the solvent.

Although the preparation of acetyl cyclosporin A is well established in the literature, it will be appreciated by those skilled in the art that protecting groups other than acetate esters may be used to protect the β-alcohol of the 1-amino acid residue of cyclosporin A. These protecting groups may include benzoate esters, substituted benzoate esters, ethers, and silyl ethers. Under certain reaction conditions, the acetate protecting group is prone to undesirable side reactions such as elimination and hydrolysis. Since benzoate esters, ethers and silyl ethers are often more resistant to such side reactions under those same reaction conditions, it is often advantageous to employ such protecting groups in place of acetate.

In step c-ii), the protected cyclosporin A of formula XIV is converted to a protected cyclosporin A aldehyde of formula II.

This step can be carried out, for example, by using ozone as an oxidizing agent followed by work-up with a reducing agent to form a protected cyclosporin A aldehyde (II). Ozonolysis step is conducted at a temperature range from about −80° C. to 0° C. The solvent used during the ozonolysis may be a lower alcohol such as methanol. The reducing agent may be a trialkylphosphine such as tributylphosphine, a triarylphosphine, a trialykylamine such as triethylamine, an alkylaminosulfide, a thiosulfate or a dialkylsulfide such as dimethylsulfide. When working with tributylphosphine as the reducing agent, the person of ordinary skill in the art will know that the reaction is dose-controlled.

Furthermore, a protected cyclosporin A aldehyde (II) can be prepared by converting the protected cyclosporinA XIV, such as acetyl cyclosporin A, to the protected cyclosporin A epoxide with a monopersulfate, preferably oxone, in the presence of a ketone, such as acetoxyacetone or diacetoxyacetone. This step is performed in an organic solvent which is inert under these reaction conditions such as acetonitrile and water. Ethylenediamintetra-acetic acid disodium salt is added to capture any heavy metal ions which might be present. The epoxidation reaction is carried out preferably at a pH over 7. This epoxidation reaction is followed by oxidative cleavage of the epoxide with periodic acid or periodate salt under acidic conditions. Optionally, the oxidation and the oxidative cleavage can be combined in a work-up procedure. These reactions have been discussed by Dan Yang, et al., in “A C₂ Symmetric Chiral Ketone for Catalytic Asymmetric Epoxidation of Unfunctionalized Olefins,” J. Am. Chem. Soc., Vol. 118, pp. 491-492 (1996), and “Novel Cyclic Ketones for Catalytic Oxidation Reactions,” J. Org. Chem., Vol. 63, pp. 9888-9894 (1998).

The use of ruthenium based oxidizing agents has been discussed by H. J. Carlsen et al. in “A Greatly Improved Procedure for Ruthenium Tetroxide Catalyzed Oxidations of Organic Compounds,” J. Org. Chem., Vol. 46, No. 19, pp 3736-3738 (1981). Carlsen et al. teach that, historically, the expense of ruthenium metal provided an incentive for the development of catalytic procedures, the most popular of which used periodate or hypochlorite as stoichiometric oxidants. These investigators found a loss of catalytic activity during the course of the reaction with the conventional use of ruthenium which they postulated to be due to the presence of carboxylic acids. The addition of nitriles to the reaction mixture, especially acetonitrile, was found to significantly enhance the rate and extent of the oxidative cleavage of alkenes in a CCl₄/H₂O/IO₄³¹ system.

For example, protected cyclosporin A aldehyde (II) can be produced from protected cyclosporin A (XIV), such as acetyl cyclosporin A, by dissolving it in a mixture of acetonitrile and water, and then adding first sodium periodate and then ruthenium chloride hydrate. The aldehyde (II) may be extracted with ethyl acetate.

### EXAMPLES

The following preparations and examples are given to enable those skilled in the art to more clearly understand and to practice the present invention. They should not be considered as limiting the scope of the invention, but merely as being illustrative and representative thereof.

Although most of the examples have been provided for the allylation-Peterson elimination sequence on acetylcyclosporin A aldehyde, other protecting groups could in principle be used. Of course, this will be limited by their compatibility with the reaction conditions as well as the possibility to remove them efficiently to provide (E)-ISA247.

### Example 1

i) Preparation of a Solution of Crude Boronic Acid of Formula III: (E)-3-(trimethylsilyl)allylboronic acid

20 g (169.8 mmol, 1 equiv.) of allyltrimethysilane (Fluka 06073) was dissolved in 140 ml of dry THF (Fluka 87368) at RT. 106.1 ml (169.8 mmol, 1 equiv.) of a 1.6 M solution of butyllithium in hexane (Acros 181270100) was added in 10 min. maintaining the temperature between 20° C. and 25° C. After 30 min. reaction, the resulting yellow to orange solution was cooled to −70° C. 40.24 ml (169.8 mmol, 1 equiv.) triisopropylborate (Fluka 92085) is added in 10 min., keeping the temperature below −60° C. After 30 min. reaction at −74° C., the cold solution was poured onto 170 ml of a 1M aqueous HCl solution. The pH was adjusted to 7-8 by further addition of of 1M HCl_{aq} (in this particular case, 26 ml). 80 ml of dichloromethane were added for extraction. The water phase was separated and re-extracted with 80 ml of dichloromethane. The organic phases were washed sequentially with 150 ml of a saturated aqueous NaCl solution, combined, dried over Na₂SO₄, filtered and concentrated under reduced pressure to ca 40 ml. The weight of the solution was adjusted to 53.6 g by addition of dichloromethane in order to obtain a ca 50% solution of boronic acid (based on the starting allyltrimethylsilane).

ii) Allylation of Acetylcyclosporin A Aldehyde

20 g (16.23 mmol, 1 equiv.) of acetylcyclosporin A aldehyde were dissolved in 100 ml of dichloromethane at RT. 25.66 g (81.15 mmol, 5 equiv.) of the previously prepared boronic acid solution (ca 50% concentration) were added in one portion. The conversion of the reaction was monitored by HPLC. Reaction was complete within 1-3 hours at RT. A ca 85:15 mixture of β-trimethylsilyalcohol diastereomers was obtained.

iii) Peterson Elimination

The Peterson elimination was conducted directly on the reaction mixture.

20 ml of THF were added and the reaction mixture was cooled to 0° C. 2.7 ml (48.69 mmol, 3 equiv.) of concentrated sulfuric acid were added. The temperature was raised to RT. After completion of the reaction (ca 1 hour), 100 ml of water were added. The organic phase was separated and washed 2 times with 50 ml water. The water phases were re-extracted sequentially with 50 ml dichloromethane. The combined organic phases were dried over Na₂SO₄, filtered and concentrated under reduced pressure at 30° C. The resulting white foam was re-dissolved in 250 ml MTBE and after a few minutes, the crystallization started. After stirring 15 min. at RT and 2 hours at 0-2° C., the suspension was filtered. The crystals were washed with 50 ml cold MTBE (−20° C.) and dried at 40-50° C. under reduced pressure to provide 19.2 g of (E)-acetyl-ISA247 as white powder in >98% isomeric purity (400 MHz ¹H NMR).

(E)-acetyl-ISA247 can be recrystallized by dissolving the solid in dichloromethane at room temperature and exchanging the solvent to MTBE (by adding MTBE, concentrating the solution to half its volume under reduced pressure at 40° C. and repeating these operation 2 to three times). The solution is cooled to room temperature and the crystallization then starts within a few minutes. The suspension is stirred at room temperature for 2 h and 30 min at 0° C. The crystals of (E)-acetyl-ISA247 are isolated after filtration, washing with MTBE and drying under reduced pressure at 40° C.

iv) Hydrolysis

Hydrolysis of E-acetyl-ISA247 provided (E)-ISA247 in 99.5% double bond isomeric purity (by HPLC).

### Example 2

i) Preparation of a Solution of the Crude Boronic Acid of Formula IIIa IIIa: (E)-3-(trimethylsilyl)allylboronic acid

6.67 ml (40.56 mmol, 10 equiv.) of allyltrimethysilane were dissolved in 33.3 ml of dry THF at RT. 25.35 ml (40.56 mmol, 10 equiv.) of a 1.6M solution of butyllithium in hexane were added in 5 min. maintaining the temperature between 14° C. and 16° C. After 60 min. reaction at RT, the resulting yellow to orange solution was cooled to −70° C. 9.614 ml (40.56 mmol, 10 equiv.) triisopropylborate were added in 10 min., keeping the temperature below −65° C. After 60 min. reaction at −70° C., the cold solution was poured onto 35 ml of a 1M aqueous HCl solution (pH=7-8). The reaction mixture was extracted with 30 ml of dichloromethane. The water phase was separated and re-extracted with 30 ml of dichloromethane. The organic phases were washed sequentially with 30 ml of a saturated aqueous NaCl solution, combined, dried over Na₂SO₄, filtered and concentrated under reduced pressure to ca 50 ml.

ii) Generation of the Boron Reagent of Formula IVa′ ((R,R)-2-[(E)-(3-trimethylsilyl-allyl)]-[1,3,2]dioxaborolane-4,5-dicarboxylic acid dimethyl ester) and Allylation of acetylcyclosporin A aldehyde

4.88 g (40.56 mmol, 10 equiv) magnesium sulfate dihydrate were added to the above boronic acid solution under stirring, followed by 7.23 g (40.56 mmol, 10 equiv.) L-(+)-dimethyltartrate. After 2 hours stirring at RT, the suspension was cooled to 0° C. and 5 g (4.056 mmol, 1 equiv.) acetyl-cyclosporin A aldehyde were added in one portion. The reaction was monitored by HPLC (ca 90% conversion after 3 hours). After 17 hours stirring at 0° C., the suspension was filtered and the filtrate was washed with 50 ml half saturated aqueous NH₄Cl solution, 50 ml half saturated aqueous NaHCO₃ solution and 50 ml half saturated aqueous NaCl solution. The aqueous phases were re-extracted with 50 ml THF and discarded. The combined organic phases were dried over Na₂SO₄, filtered and concentrated at 40° C. under reduced pressure to provide 11.1 g of a ca 75:25 mixture of β-trimethylsilyalcohol diastereomers as a light yellow oil.

iii) Peterson Elimination

The crude β-trimethylsilyalcohol diastereomers mixture (11 g, maximum 4.056 mmol) was dissolved in 25 ml THF. 0.679 ml (12.16 mmol, 3 equiv.) concentrated sulfuric were added dropwise maintaining the temperature between 20° C. and 25° C. After 2 hours at RT, 50 ml half saturated aqueous NaCl solution were added. The resulting mixture was extracted twice with 50 ml MTBE. The organic phases were washed with 50 ml of a half saturated aqueous NaCl solution, combined, dried over Na₂SO₄ and concentrated under reduce pressure at 40° C. The resulting crude E-acetyl-ISA247 was re-dissolved in 20 ml dichloromethane and concentrated under reduced pressure. The crude product was dissolved in 60 ml MTBE. The crystallization started within 10 min. The suspension was stirred for an additional 15 min. at RT and 2 hours at −10° C. The crystals were isolated by filtration, washed with 20 ml cold MTBE (−20° C.) and dried under reduced pressure to provide 3.6 g of (E)-acetyl-ISA247 in ca 98% isomeric purity by NMR.

### Example 3

i) Preparation of Diethanolamine Complex of Formula V: 2-(E-(3-trimethylsilyl-allyl))-[1,3,6,2]dioxazaborocane

50 g (424.5 mmol, 1 equiv.) allyltrimethylsilane were charged in the reaction vessel followed by 150 ml THF. To the clear colorless solution were added dropwise over 15 min., 165.1 ml (445.7 mmol, 1.05 equiv.) of a 2.7M butyllithium solution in heptane, maintaining the temperature between 20° C. and 26° C. After 2 hours reaction at RT, the orange solution was cooled to −78° C. 105.7 ml (445.8 mmol, 1.05 equiv.) triisopropylborate were added dropwise over 20 min., maintaining the temperature below −60° C. After 1 hour at −70° C., the reaction mixture was poured onto 250 ml of a 2M aqueous hydrochloric acid solution (resulting pH: 5-6). After 10 min. stirring, the water phase was separated and discarded. 42.4 g (403.3 mmol, 0.95 equiv) diethanolamine were added to the organic phase. The solution was stirred for 60 min. at RT. 750 ml heptane were added. The biphasic emulsion was partially concentrated at 40° C. (ca 750 ml solvent distilled) under reduced pressure. A white precipitate appeared and the suspension was stirred for 2 hours at RT. The suspension was filtered. The white solid was washed with 125 ml heptane and dried at 40° C. under reduced pressure overnight to provide 85.7 g of the diethanolamine complex of formula V.

¹H NMR (DMSO, in ppm rel. to TMS): 6.5 (br s, 1H), 6.15 (dt, 1H), 5.32 (d, 1H), 3.7 (m, 2H), 3.55 (m, 2H), 2.95 (m, 2H), 2.72 (m, 2H), 1.29 (d, 2H), 0 (s, 9H).

¹¹B NMR (DMSO, rel. to external ref. BF₃.Et₂O): 11.1 (br, s); Microanalysis: (contains 0.11 equiv. H₂O by Karl-Fischer titration); Calcd: C,52.43%, H, 9.71%, N, 6.12%, B, 4.72%, Si, 12.27%. Found: C, 52.04%, H, 9.63%, N, 6.36%, B, 4.79%, Si, 11.3%;

ii) Allylation

7.375 g (32.46 mmol, 2 equiv.) of diethanolamine complex of formula V, 20 g (16.23 mmol, 1 equiv.) acetylcyclosporin A aldehyde and 80 ml dichloromethane were charged in the reaction vessel at RT. 40 ml water and 2.79 ml (48.69 mmol, 3 equiv.) acetic acid were added under stirring. After 10 min. stirring, a clear biphasic mixture was obtained. The reaction was monitored by HPLC.

iii) Peterson Elimination

After overnight reaction, the organic layer was separated and the water phase was discarded. 50 ml THF were added to the organic phase. The solution was concentrated under reduced pressure at 30° C. to half its volume. 100 ml THF were added and the solution was concentrated to 80 ml. The volume was adjusted to 100 ml with THF and the solution was cooled to 0-2° C. 1.812 ml (32.46 mmol, 2 equiv.) concentrated sulfuric acid were added dropwise over 5 min., maintaining the temperature below 5° C. After addition, the reaction cooling bath was removed and the temperature was raised to RT. After 4 hours reaction, 40 ml water were added followed by 20 ml MTBE. The aqueous layer was separated and discarded. The organic phase was washed with 40 ml NaHCO_{3 aq}, 20 ml saturated NaCl_{aq}, 40 ml saturated NaCl_{aq}, dried over Na₂SO₄, filtered and concentrated at 40° C. under reduced pressure. The crude E-acetyl-ISA247 was re-dissolved in 200 ml MTBE and crystallization started within a few minutes. After 15 min. at RT and 2.5 hours at 0° C., the suspension was filtered, the crystals were washed with 50 ml MTBE and dried at 50° C. under reduced pressure to give 18.45 g of (E)-acetyl-ISA247 as a white powder (>98% isomeric purity by NMR).

iv) Hydrolysis

This crude product was hydrolyzed to give (E)-ISA247 in 99% isomeric purity by HPLC.

### Example 4

i) Allylation

10.02 g (44.1 mmol, 2 equiv.) diethanolamine complex of formula V obtained by the method described in Example 3, i), and 30 g (22.05 mmol, 1 equiv.) of acetyl-cyclosporin A aldehyde were charged in the reaction vessel. 36 ml acetic acid were added at RT. A clear solution was obtained after 15 min. stirring at RT. The reaction was monitored by HPLC.

ii) Peterson Elimination

After ca 6 hours reaction at RT, 60 ml formic acid were added, maintaining the temperature below 30° C. The clear light yellow solution was stirred overnight at RT. 18 ml dichloromethane and 300 ml MTBE were added followed by 180 ml of a 10% NaCl_{aq} solution. The aqueous phase was separated and discarded. The organic phase was washed with 180 ml water, 300 ml 2M aqueous NaOH and 90 ml water. The organic phase was concentrated at RT under reduced pressure. The crystallization started and the suspension was diluted by addition of 300 ml MTBE and concentrated to ca 330 ml. After stirring for 3 hours at RT and 1 hour at 0-2° C., the white suspension was filtered. The crystals were washed with 50 ml MTBE and dried at 50° C. under reduced pressure to give 27.4 g of (E)-acetyl-ISA247 as a white powder in >98% double bond isomeric purity by NMR.

iii) Hydrolysis

This product was hydrolyzed to give (E)-ISA247 in 99.6% double bond isomeric purity by HPLC.

### Example 5

1 g (0.82 mmol, 1 equiv.) acetylcyclsoporine A aldehyde were dissolved in 10 ml dichloromethane followed by 369 mg (1.62 mmol, 2 equiv.) diethanolamine complex of formula V obtained by the method described in Example 3, i). The turbid solution was cooled to −40° C. 180 μl (369 mmol, 2 equiv.) boron trifluoride etherate were added keeping the temperature below −40° C. After 1 hour at −40° C., the cooling bath was removed and the reaction mixture was warmed up to RT. After 50 min. reaction at RT, 15 ml of a 5% aqueous NaHCO₃ solution were added. The aqueous phase was separated and re-extracted with 15 ml dichloromethane. The combined organic phases were dried over MgSO₄, filtered and concentrated under reduced pressure at 40° C. to give 0.99 g of (E)-acetyl-ISA247 in >95% double bond isomeric purity (NMR) as a white foam.

### Example 6

i) Preparation of the Pinacol Complex of Formula VII: 4,4,5,5-tetramethyl-2-(E-(3-trimethylsilyl-allyl))-[1,3,2]dioxaborolane

20 g (169.8 mmol, 1 equiv.) of allyltrimethylsilane were dissolved in 60 ml THF. 69.18 ml (186.8 mmol, 1.1 equiv.) of a 2.7M butyllithium solution in heptane were added dropwise over 10 min. maintaining the temperature between 20° C. and 26° C. After 2 hours reaction at RT, the yellow solution was cooled to −78° C. 42.26 ml (178.3 mmol, 1.05 equiv.) of triisopropylborate were added dropwise over 10 min., maintaining the temperature below −65° C. After 1 hour reaction, the reaction mixture was poured onto 100 ml of a 2M aqueous hydrochloric acid solution (resulting pH, 6-7). 20 ml dichloromethane were added and the water phase was separated and discarded. The organic layer was dried over MgSO₄, filtered and concentrated to about 100 ml. 20.48 g (169.8 mmol, 1.0 equiv.) pinacol were added and the resulting solution was stirred for 18 hours at RT. The reaction mixture was concentrated at 40° C. under reduced pressure and the resulting oil was distilled at 43-50° C. under 0.2 mbar pressure to give 37.2 g of a colorless oil.

ii) One-Pot Allylation/Peterson Elimination

20 g (15.06 mmol, 1 equiv.) of acetylcyclosporin A aldehyde and 5.427 g (22.59 mmol, 1.5 equiv.) pinacol complex obtained in i) and 30 ml acetic acid were charged in the reaction vessel at RT under stirring. 30 ml of formic acid were added under water bath cooling, maintaining the temperature between 20-22° C. After 2 hours reaction at RT, 12 ml dichloromethane and 200 ml MTBE were added followed by 120 ml of a 10% aqueous NaCl solution. The water phase was separated and discarded. The organic phase was washed with 120 ml water, 204 ml 2M aqueous NaOH solution and 60 ml water. The organic phase was concentrated at 30° C. until the crystallization started. 200 ml MTBE were added and the suspension was concentrated to ca 220 ml. After stirring at RT for 2 hours and for 1 hour at 0-2° C., the suspension was filtered. The solid was washed with 30 ml MTBE and dried at 50° C. under reduced pressure to provide 18 g of (E)-acetyl-ISA247 as a white powder in >98% double bond isomeric purity (by NMR).

iii) Hydrolysis

This product was hydrolyzed to give (E)-ISA247 in 99.7% double bond isomeric purity by HPLC.

### Example 7

2 g (1.623 mmol, 1 equiv.) acetylcyclosporin A aldehyde and 779.8 mg (3.246 mmol, 2 equiv.) pinacol boronate obtained by the method described in Example 6, i) were dissolved in 20 ml dichloromethane. The solution was cooled to −70° C. and 1.28 ml (10.22 mmol, 6.30 equiv.) borontrifluoride etherate were added. After 30 min. at −70° C., the reaction mixture was slowly warmed up to 0° C. and reaction was continued for 60 min. at 0° C. 20 ml water were added. The organic phase was separated, washed with 20 ml of a 5% aqueous NaHCO₃ solution, dried over MgSO₄, filtered and concentrated at 40° C. under reduced pressure to give 2.1 g of (E)-acetyl-ISA247 in >95% double bond isomeric purity (NMR) as a white foam.

### Example 8

i) Preparation of Allyltrifluoroborate Reagent: Potassium B-(E-(3-trimethylsilyl-allyl))-trifluoroborate

5 g (21.35 mmol, 1 equiv) of diethanolamine complex obtained by the method described in Example 3, i), 20 ml dichloromethane, 20 ml water and 2.44 ml (42.70 mmol, 2 equiv.) acetic acid were charged in the reaction vessel under stirring at RT. After 30 min. stirring, the water phase was separated and discarded. 20 ml methanol were added to the organic phases and the solution was concentrated at 40° C. under reduced pressure to 5-10 ml. 40 ml methanol were added followed by 3.34 g (42.70 mmol, 2 equiv.) KHF₂. After 60 min. stirring at RT, the remaining solid was filtered and discarded. The filtrate was concentrated under reduced pressure at 40° C. to ca 25 ml. The solution was cooled to 0-2° C. and a white suspension was obtained. After 30 min. at 0-2° C., the suspension was filtered and the solid was washed with cold methanol (−20° C.) and dried under reduced pressure at 40° C. to give 3.4 g of a white powder.

¹H NMR (DMSO, 6 in ppm rel. to TMS): 6.15 (1H, dt), 5.15 (1H, d), 3.5 (2H, br s water), 1.1 (2H, m), 0 (9H, s).

¹B NMR (6 in ppm rel. to BF₃.Et₂O external ref.): 3.8 (q); Microanalysis: C₁₅H₁₃F₃BKSi (contains 1.08 equiv. H₂0 by Karl-Fischer titration and 0.5 equiv. KF): Calcd: C, 26.83%, H, 5.65%, F, 24.78%, B, 4.02%, K, 21.8%, Si, 10.5%. Found: C, 26.33%, H, 5.74%, F, 24.71%, B, 3.89%, K 22%, Si, 9.93%.

ii) Allylation

2 g (1.623 mmol, 1 equiv.) acetylcyclosporin A aldehyde were dissolved in 10 ml dichloromethane. 10 ml water were added, followed by 735 mg (3.246 mmol, 2 equiv.) of trifluoroborate obtained in i). After 2 hours stirring at RT, the organic phase was separated and the water phase discarded.

iii) Peterson Elimination

5 ml THF were added to the organic phase and the solution was cooled to 0-2° C. 181 μl (3.246, 2 equiv.) concentrated sulfuric acid were added. The reaction mixture was warmed up to RT. After stirring overnight, 20 ml water were added. The aqueous layer was separated and discarded. The organic phase was washed with 20 ml of 5% aqueous NaHCO₃ solution, dried over MgSO₄, filtered and concentrated under reduced pressure at 40° C. to give 2 g of (E)-acetyl-ISA247 as a white foam in >98% double bond isomeric purity (by NMR).

### Example 9

2 g (1.623 mmol, 1 equiv.) acetylcyclosporin A aldehyde and 735 mg (3.246 mmol, 2 equiv.) trifluoroborate obtained by the method described in Example 8, i) and 20 ml dichloromethane were charged in the reaction vessel. The suspension was cooled to −70° C. and 1.28 ml (10.22 mmol, 6.3 equiv.) borontrifluoride etherate were added. After 60 min. at −70° C., 20 ml water were added. The organic phase was separated, washed with 20 ml of a 5% aqueous NaHCO₃ solution, dried over MgSO₄, filtered and concentrated at 40° C. under reduced pressure to give 2.0 g of (E)-acetyl-ISA247 in >98% double bond isomeric purity (NMR) as a white foam.

### Example 10

i) Allylation by an Allyltitanium Reagent

2.67 ml (16.23 mmol, 10 equiv.) allyltrimethylsilane were dissolved in 6 ml THF. 10.14 ml (16.23 mmol, 10 equiv.) of a 1.6M butyllithium solution in hexane were added dropwise maintaining the temperature between 14-20° C. After 30 min. at 26° C., the orange solution was cooled down to −75° C. 4.8 ml (16.23 mmol, 10 equiv.) titanium tetraisopropoxide were added dropwise over 10 min., maintaining the temperature below −68° C. After 1 hour reaction at −77° C., 2 g (1.623 mmol, 1 equiv.) of acetyl cyclosporin A aldehyde in solution in 6 ml THF were added dropwise maintaining the temperature below −72° C. The reaction mixture was stirred for 2 hours at −76° C. The temperature was slowly raised to −40° C. and the stirring was continued for a further 2 hours at −40° C. The reaction mixture was poured onto a mixture consisting of 32.5 ml of a 1M aqueous HCl solution and 20 ml MTBE. 16.2 ml of a 1M aqueous HCl solution and 25 ml water were added. The aqueous layer was separated and re-extracted with 25 ml MTBE. The organic layers were washed with 30 ml of 0.5 M HClaq, combined, dried over Na₂SO₄, filtered and concentrated under reduced pressure at 40° C. to give 2.26 g of crude mixture of diastereomeric anti β-trimethylsilyalcohols as a white foam.

ii) Peterson Elimination

The crude product was dissolved in 11.15 ml THF and 268 μl concentrated sulfuric acid were added. The reaction mixture was heated at 33° C. for 1.5 hour and then cooled to RT. 22 ml water were added and the reaction mixture was extracted with 22 ml MTBE. The aqueous phase was re-extracted with 11 ml MTBE. The organic layer were washed with 11 ml water, combined, dried over Na₂SO₄, filtered and concentrated at 40° C. under reduced pressure to give 1.89 g of crude (E)-acetyl-ISA247 as a beige powder. The crude product was re-dissolved in 20 ml MTBE at RT. The crystallization started within a few minutes. The suspension was stirred 30 min. at RT, 45 min. at −10° C. and was filtered. The solid was washed with cold MTBE and dried at 40° C. under reduced pressure to give 1.02 g of (E)-acetylISA247 as a white powder in ca 98% double bond isomeric purity (NMR).

### Example 11

i) Allylation by an Allyltitanium Reagent

1.87 g (15.85 mmol, 10 equiv.) of allyltrimethylsilane were dissolved in 20 ml of THF at RT. 5.87 ml (15.85 mmol, 10 equiv.) of a 2.7 M solution of butyllithium in heptane were added dropwise over 5 min., keeping the temperature between 16° C. and 20° C. After 1 hour stirring at RT, the yellow to orange solution was cooled to −76° C. A solution of 4.22 g (15.85 mmol, 10 equiv.) of titanium chlorotriisopropoxide in 10 ml THF was added dropwise over 4 min., keeping the temperature below −60° C. The resulting brown-red solution was stirred for 30 min. at −75° C. A solution of 2 g (1.585 mmol, 1 equiv.) of acetylcyclosporin A aldehyde in 10 ml of THF was added dropwise over 5 min. maintaining the temperature below −60° C. After 30 min. at −75° C., the cooling bath was removed and the temperature was raised to −10° C. over ca 15 min. The reaction mixture was added to a biphasic mixture consisting of 40 ml MTBE and 35 ml of a 2M aqueous HCl solution. The aqueous layer was separated and discarded. The organic phase was washed with 24 ml of 1M aqueous HCl solution, 15 ml of a 10% aqueous NaCl solution, 15 ml of a half saturated aqueous NaCl solution, dried over Na₂SO₄, filtered and concentrated under reduced pressure to provide 2 g of the crude mixture of anti β-trimethylsilylalcohol diastereomers as a solidifying oil.

ii) Peterson Elimination

The crude product was dissolved in 8 ml THF at RT. The solution was cooled to 0-5° C. and 200 μl of concentrated sulfuric acid were added dropwise. The temperature was raised to RT and the reaction mixture was stirred 10 hours. 40 ml MTBE and 15 ml of water were added. The water phase was separated and discarded. The organic phase was washed with 15 ml of a 5% aqueous NaHCO₃ solution, 15 ml of a half saturated aqueous NaCl solution, dried over Na₂SO₄, filtered and concentrated under reduced pressure to give 1.8 g of crude E-acetyl-ISA247. The crude diene was dissolved in 20 ml dichloromethane. 20 ml MTBE were added, and the solution was concentrated at 40° C. under reduced pressure to half its volume. The last two operations were repeated three times in order to exchange the solvent from dichloromethane to MTBE. The solution was cooled to RT and the crystallization started within a few minutes. The suspension was stirred 2 hours at RT and 30 min. at 0° C. The suspension was filtered. The solid was washed with 15 ml MTBE and dried under reduced pressure at 40° C. to give 1.1 g of E-acetyl-ISA247 in >95% double bond isomeric purity (NMR), as a white powder.

### Example 12

i) Allylation by Allylaluminum Reagent:

1.87 g (15.85 mmol, 10 equiv.) of allyltrimethylsilane were dissolved in 20 ml of THF at RT. 5.87 ml (15.85 mmol, 10 equiv.) of a 2.7M solution of butyllithium in heptane were added dropwise over 5 min., keeping the temperature between 20° C. and 25° C. After 1 hour stirring at RT, the yellow to orange solution was cooled to −75° C. 8.6 ml (15.85 mmol, 10 equiv.) of a 25% solution of diethylaluminum chloride in toluene were added over 10 min., keeping the temperature below −55° C. The resulting clear colorless solution was stirred for 30 min. at −75° C. A solution of 2 g (1.585 mmol, 1 equiv.) of acetylcyclosporin A aldehyde in 10 ml of THF was added dropwise over 5 min. maintaining the temperature below −60° C. After 30 min. at −75° C., the cooling bath was removed and the temperature was raised to −10° C. over 15 min. The reaction mixture was slowly added (under cold water bath cooling 10° C.) to a biphasic mixture consisting of 40 ml MTBE and 35 ml of a 1M aqueous HCl solution. The aqueous layer was separated and discarded. The organic phase was washed with 35 ml of 1M aqueous HCl solution, 25 ml of water, 25 ml of a saturated aqueous NaCl solution, dried over Na₂SO₄, filtered and concentrated under reduced pressure to provide 2 g of the crude mixture of anti β-trimethylsilylalcohol diastereomers as a solidifying oil.

ii) Peterson Elimination

The crude product was dissolved in 10 ml THF at RT. The solution was cooled to 0-5° C. and 200 μl of concentrated sulfuric acid were added dropwise. The temperature was raised to RT and the reaction mixture was stirred overnight. 40 ml MTBE and 15 ml of water were added. The water phase was separated and discarded. The organic phase was washed with 15 ml water, 15 ml of a 5% aqueous NaHCO₃ solution, 15 ml of a half saturated aqueous NaCl solution, filtered and concentrated under reduced pressure to give 1.8 g of crude E-acetyl-ISA247. The crude diene was redissolved in 35 ml of MTBE. The crystallization started within a few minutes. The suspension was stirred 2 hours at RT and 30 min. at 0° C. The suspension was filtered. The solid was washed with 15 ml MTBE and dried under reduced pressure at 40° C. to give 1 g of E-acetyl-ISA247 in >95% double bond isomeric purity (NMR), as a white powder.

### Example 13

### Preparation of a Solution of Boron Reagent of Formula IIIa: (E)-3-(trimethylsilyl)allylboronic acid

2 g (8.8 mmol, 1 equiv.) of diethanolamine complex of formula V as prepared in Example 3-i) was dissolved in 16 ml of d₂-dichloromethane (deuterated dichloromethane). 759 μl (13.2 mmol, 1.5 equiv.) of acetic acid were added, followed by 4 ml of water. The biphasic mixture was stirred for 20 min. at RT to give a light yellow clear biphasic mixture. Stirring was stopped, the water phase was separated and discarded. The organic phase (16 ml volume) consisted in a solution of boronic acid of formula IIIa as evidenced by ¹¹B NMR and ¹H NMR.

¹¹B NMR (δ in ppm relative to BF₃.Et₂O as external reference): 31.7.

¹H NMR (in CD₂C₁₂, δ in ppm relative to TMS): 6.1 (1H, dt), 5.6 (1H, d), 1.77 (2H, d), 0 (9H, s).

### Example 14

### Preparation of a Solution of Boron Reagent of Formula IVa′: (R,R)-2-[(E)-(3-trimethylsilyl-allyl)[-]1,3,2]dioxaborolane-4,5-dicarboxylic acid dimethyl ester

To 4 ml (2.2 mmol, 1 equiv.) of the solution of the boronic acid of formula IIIa, prepared as described in Example 13, were added 396 mg (2.2 mmol, 1 equiv.) of L-(+)-dimethyltartrate and 265 mg (2.2 mmol, 1 equiv.) of magnesium sulfate dihydrate. The suspension was stirred for 40 min. at RT and was filtered. The filtrate was analyzed by NMR and was shown to contain, as main product the boronate ester of formula IVa′ as evidenced by the appearance of consistent ¹¹B and ¹H NMR signals.

¹¹B NMR (in CD₂Cl₂, δ in ppm relative to BF₃.Et₂O as external reference): 34.2.

¹H NMR (in CD₂Cl₂, δ in ppm relative to TMS): 6.07 (1H, dt), 5.64 (1H, d), 1.93 (2H, d), 0 (9H, s).

### EXAMPLE 15

### Hydrolysis of E-acetyl-ISA247 to E-ISA247

15 g (11.94 mmol, 1 equiv.) of E-acetyl-ISA247 were dissolved at RT in 270 ml of methanol. A solution of 14.85 g (107.5 mmol, 9 equiv.) of potassium carbonate in 60 ml of water was added keeping the temperature below 27° C. The white suspension was heated to 30° C. The reaction was monitored by HPLC. After 22 hours of reaction, the methanol was evaporated at 40° C. under reduced pressure. The residue was taken up in 150 ml of ethylacetate. The water phase was separated and discarded. The organic phase was washed with 45 ml of a 5% aqueous solution of citric acid and 45 ml of a half saturated aqueous NaCl solution, dried over Na₂SO₄, filtered and concentrated under reduced pressure at 40° C. to give 15.1 g of E-ISA247 (85% assay). Purification can be performed by chromatographic techniques like preparative HPLC.

### Example 16

i) Preparation of the Pinacol Complex of Formula VII: 4,4,5,5-tetramethyl-2-(E-(3-trimethylsilyl-allyl))-[1,3,2]dioxaborolane

100 g allyltrimethylsilane (1 equiv.) were charged in reactor 1 followed by 300 ml THF. The solution was cooled to 10-15° C. and 374 ml of 2.5M Butyllithium solution in hexane (1.1 equiv.) were added keeping the temperature below 25° C. (over ca 30 min.). After 1-2 hours at 20-25° C., the yellow to orange solution was cooled to −50° C. 173 g Triisopropylborate (1.05 equiv.) were added dropwise keeping the temperature below −40° C. (over ca 30-45 min.). The dropping funnel was washed with 25 ml THF. After 30 min. to 1 hour at −50° C. to −40° C., a solution of 102.4 g of pinacol (1 equiv.) in 100 ml THF was added, keeping the temperature below −30° C. The dropping funnel was washed with 25 ml THF. After 30 min. at −50° C. to −30° C., the content of reactor 1 was poured, under stirring, onto a mixture of 61.2 g AcOH (1.2 equiv.) and 250 ml water (contained in reactor 2), keeping the temperature between 0-25° C. Reactor 1 was washed with 50 ml THF.

Stirring was discontinued in reactor 2, the aqueous phase was separated and discarded. The organic layer was washed with 250 ml water. The organic phase was concentrated to ca 500 ml (Ti=20-40° C., 150-200 mbar). 500 ml Toluene were added and the organic phase was concentrated to 500 ml (Ti=40-50° C., Tj=50° C., 150-40 mbar). 500 ml Toluene were added and the organic phase was concentrated until constant volume (Ti=40-50° C., Tj=50° C., 150-10 mbar) to provide crude pinacol complex in >90% yield. The complex could be used directly in the allylation-Peterson elimination sequence or could be distilled under reduced pressure (Ti=ca 65° C., Tdest=ca 50° C., P=0.05-0.15 mbar).

ii) One-Pot Allylation-Peterson Elimination

40 g acetyl protected CsA-Aldehyde were charged in a feed vessel followed by 80 ml isopropyl acetate. The suspension was transferred to the reactor. The feed vessel was washed with 50 ml acetic acid which was transferred to the reactor. A clear solution was then obtained. Pinacol complex (1.25-1.5 equiv.) was added. The clear solution was heated to 40° C. 50 ml formic acid were added. After completion of the allylation and Peterson elimination as evidenced by HPLC analysis (after ca 15-20 hours), 246 ml isopropyl acetate were added. The reaction mixture was washed twice with 200 ml water, 300 g of 2M aqueous KOH solution (pH of the aqueous phase set between 5-8, if necessary with additional KOH solution) and 200 ml of 5% aqueous ammonium formate. The organic phase was concentrated to ca 120 ml (Ti=ca 40° C., ca 200 mbar) and was diluted with 300 ml methanol. The organic phase was concentrated to ca 120 ml (Ti=ca 40° C., 200 mbar) and was diluted with 240 ml methanol (Ti=ca 40° C., 200 mbar). The organic phase was concentrated to ca 280 ml. 130 ml water were added over ca 60 min. at 20-25° C. The resulting white suspension was stirred 60 min. at room temperature. The solid was isolated by filtration, washed twice with 52 ml of a water/methanol mixture, dried under vacuum (T=50° C.) until constant weight to provide E-acetyl-ISA247 (ca 35 g).

What is claimed is:

1. A process for the preparation of a cyclosporin A analog of formula I 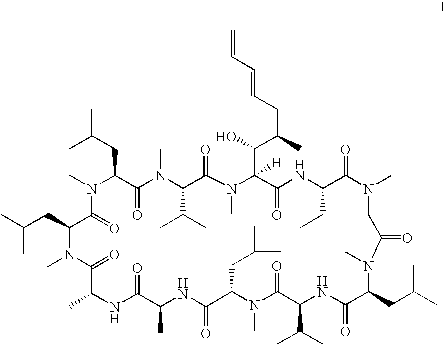
comprising
(a) allylating a compound of formula II 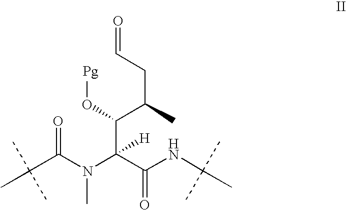
wherein Pg is a protecting group and the dotted lines mean that the remainder of the compound has the same structure as that of the compound of formula I with a compound of formula III 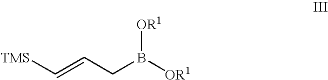
wherein R¹ is hydrogen, C₁₋₈alkyl, or C₃₋₈cycloalkyl and/or when R¹ is hydrogen, a trimer thereof in dichloromethane or toluene to form a compound of formula XI 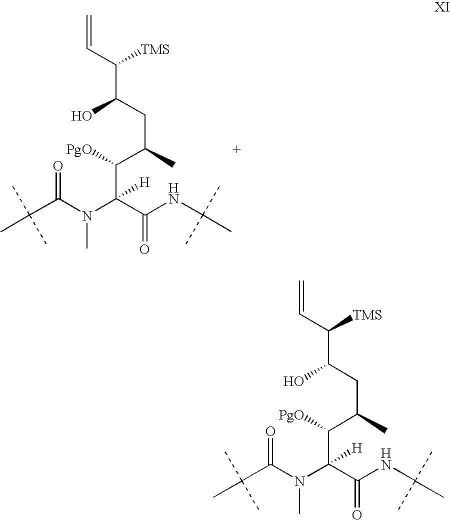
and
(b) converting the compound of formula XI to the cyclosporin A analog of formula I.

2. The process according to claim 1, wherein conversion of the compound of formula XI to the cyclosporin A analog of formula I comprises
(b-i) converting the compound of formula XI 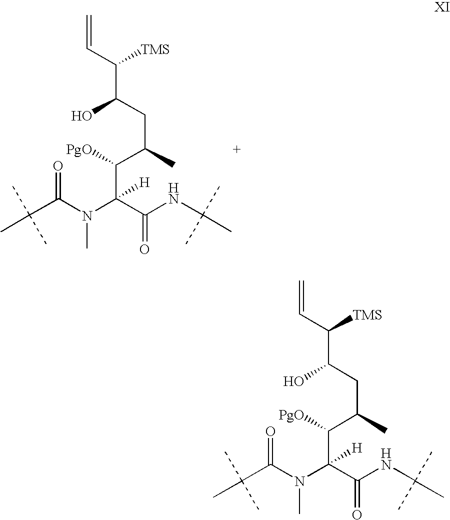
to a compound of formula XII 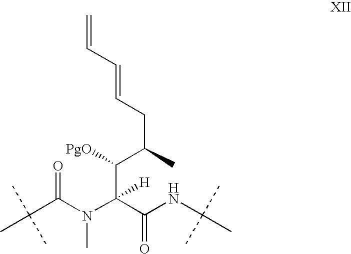
wherein Pg is as defined in claim 1, under acidic conditions, and
(bii) converting the PgO group of the compound of formula XII to a hydroxyl group.

3. The process according to claim 1 wherein Pg is an acetyl group.

4. The process according to claim 1 wherein step (a) is conducted in the presence of tartrates.

5. A process for the preparation of a cyclosporin A analog of formula I 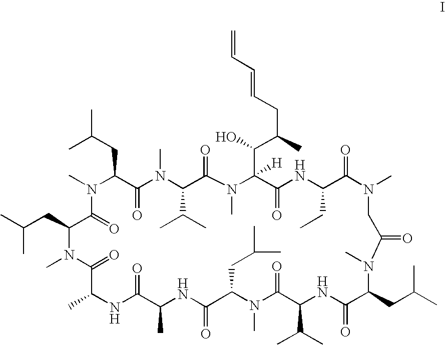
comprising
(a) allylating a compound of formula II 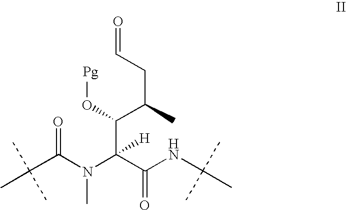
wherein Pg is a protecting group and the dotted lines mean that the remainder of the compound has the same structure as that of the compound of formula I with a compound of formula IV 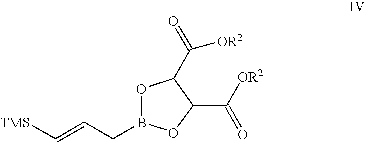
wherein R² is C₁₋₈alkyl or C₃₋₈cycloalkyl in dichloromethane or toluene to form a compound of formula XI 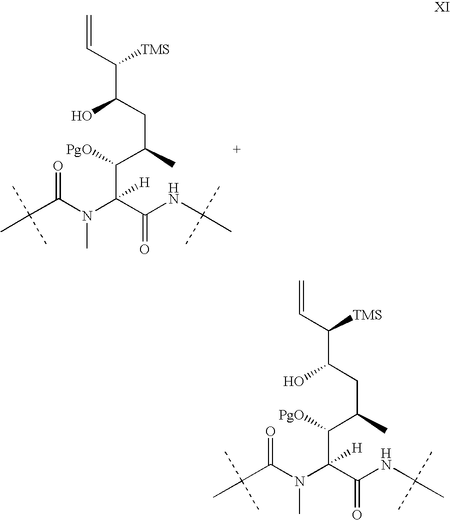
and
(b) converting the compound of formula XI to the cyclosporin A analog of formula I.

6. The process according to claim 5, wherein conversion of the compound of formula XI to the cyclosporin A analog of formula I comprises
(b-i) converting the compound of formula XI 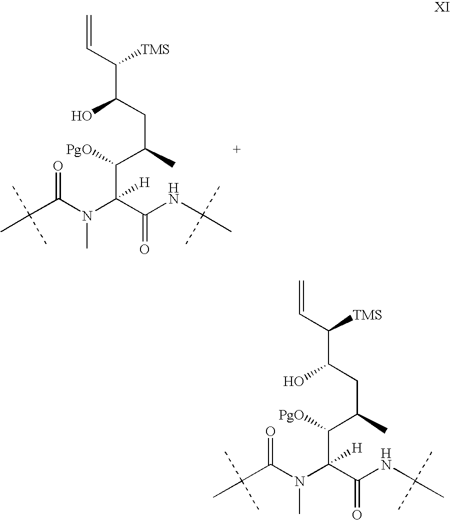
to a compound of formula XII 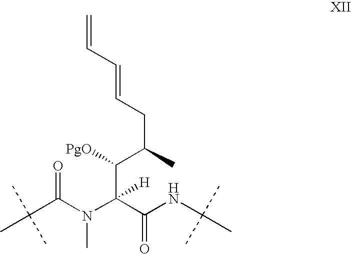
wherein Pg is as defined in claim 5, under acidic conditions, and
(bii) converting the PgO group of the compound of formula XII to a hydroxyl group.

7. The process according to claim 5 wherein Pg is an acetyl group.

8. The process according to claim 5 wherein the process is conducted in the presences of tartrates.

9. A process for the preparation of a cyclosporin A analog of formula I comprising 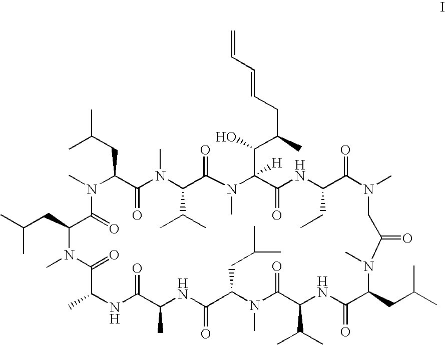
(a) allylating a compound of formula II 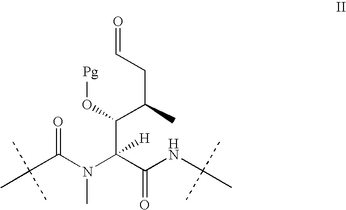
wherein Pg is a protecting group and the dotted lines mean that the remainder of the compound has the same structure as that of the compound of formula I with a compound of formula V 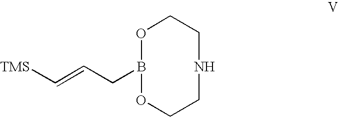
in water/dichloromethane or water/toluene to form a compound of formula XI 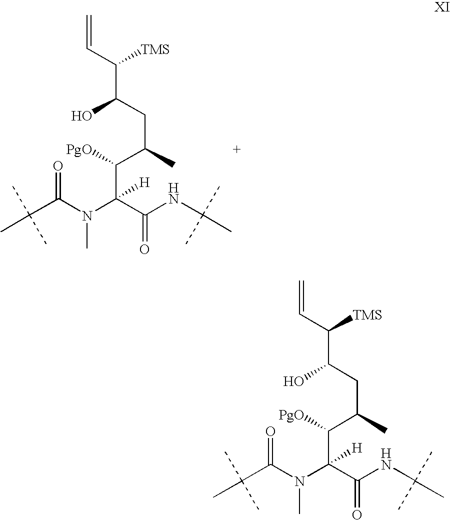
and
(b) converting the compound of formula XI to the cyclosporin A analog of formula I.

10. The process according to claim 9, wherein conversion of the compound of formula XI to the cyclosporin A analog of formula I comprises
(b-i) converting the compound of formula XI 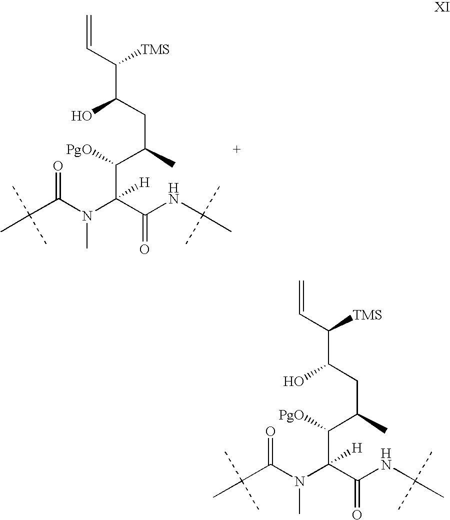
to a compound of formula XII 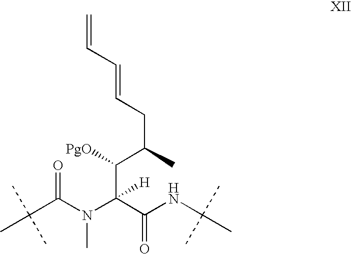
wherein Pg is as defined in claim 9, under acidic conditions, and
(bii) converting the PgO group of the compound of formula XII to a hydroxyl group.

11. The process according to claim 9 wherein Pg is an acetyl group.

12. A process for the preparation of a cyclosporin A analog of formula I 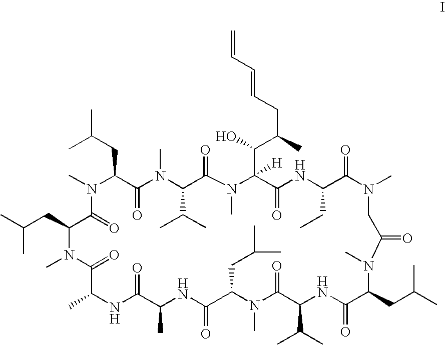
comprising
(a) allylating a compound of formula II 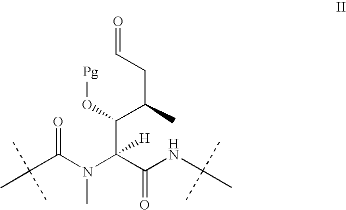
wherein Pg is a protecting group and the dotted lines mean that the remainder of the compound has the same structure as that of the compound of formula I with a compound of formula V 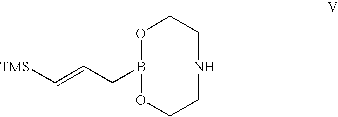
in the presence of BF₃.Et₂O, formic acid, acetic acid, or tartrate esters to form a compound of formula XI 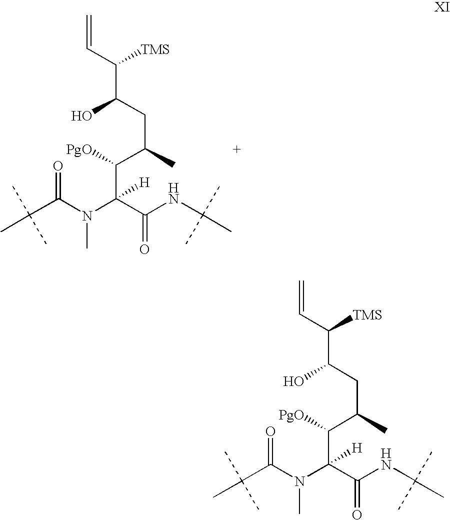
and
(b) converting the compound of formula XI to the cyclosporin A analog of formula I.

13. The process according to claim 12, wherein conversion of the compound of formula XI to the cyclosporin A analog of formula I comprises
(b-i) converting the compound of formula XI 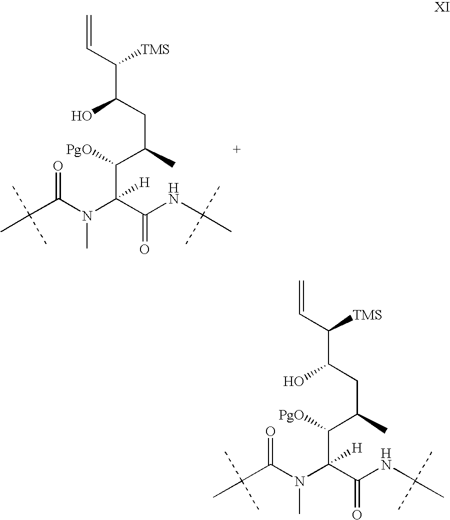
to a compound of formula XII 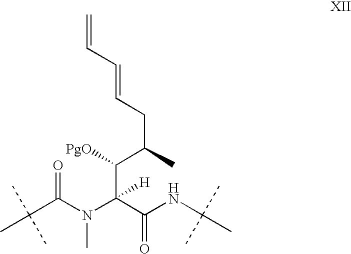
wherein Pg is as defined in claim 12, under acidic conditions, and
(bii) converting the PgO group of the compound of formula XII to a hydroxyl group.

14. A process according to claim 13 wherein steps (a) and (b-i) are conducted in dichloromethane or tetrahydrofuran and in the presence of BF₃.Et₂O.

15. The process according to claim 12 wherein Pg is an acetyl group.

16. A process for the preparation of a cyclosporin A analog of formula I 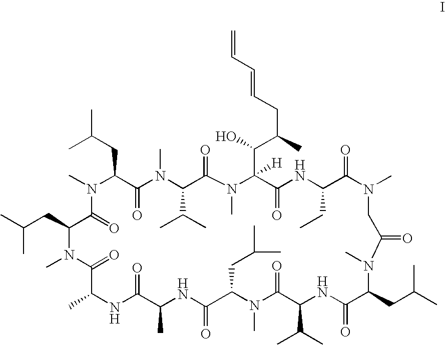
comprising
(a) allylating a compound of formula II 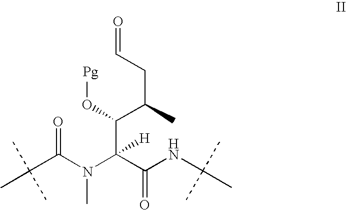
wherein Pg is a protecting group and the dotted lines mean that the remainder of the compound has the same structure as that of the compound of formula I with a compound of formula V 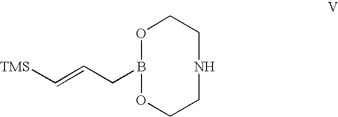
in acetic acid, formic acid, or a mixture of acetic acid and formic acid and in one or two cosolvents selected from the group consisting of dichloromethane and tetrahydrofuran
to form a compound of formula XI 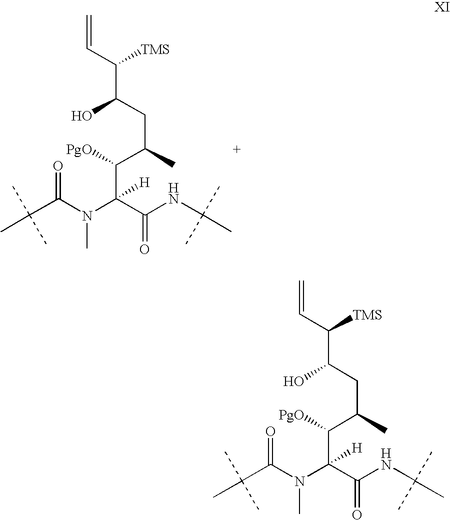
and
(b) converting the compound of formula XI to the cyclosporin A analog of formula I.

17. The process according to claim 16, wherein conversion of the compound of formula XI to the cyclosporin A analog of formula I comprises
(b-i) converting the compound of formula XI 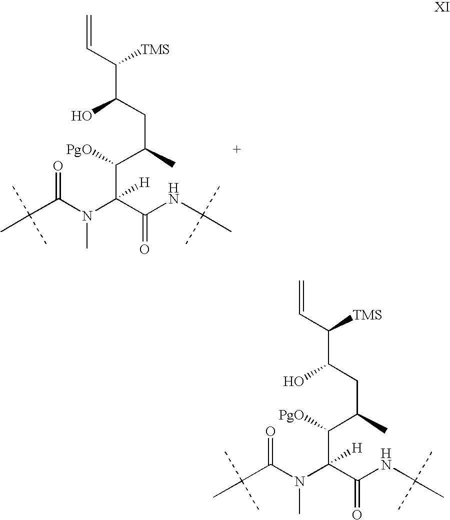
to a compound of formula XII 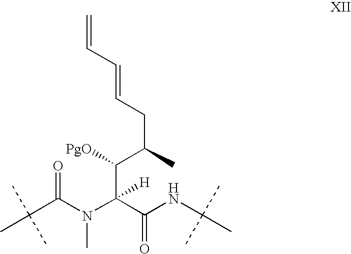
wherein Pg is as defined in claim 16 under acidic conditions, and
(bii) converting the PgO group of the compound of formula XII to a hydroxyl group.

18. The process according to claim 17 wherein step (a) is conducted in acetic acid and step (b-i) is conducted by the addition of formic acid to the reaction mixture.

19. The process according to claim 17 wherein steps (a) and (b-i) are conducted in formic acid or acetic acid/formic acid.

20. The process according to claim 16 wherein Pg is an acetyl group.

21. A process for the preparation of a cyclosporin A analog of formula I 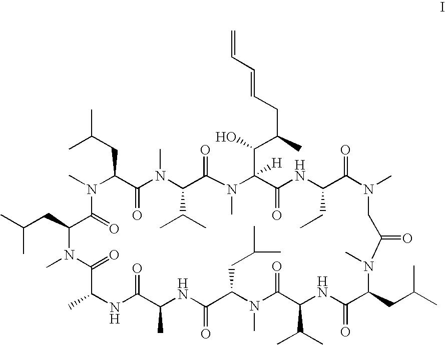
comprising
(a) allylating a compound of formula II 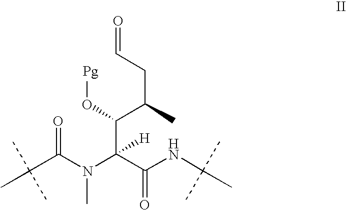
wherein Pg is a protecting group and the dotted lines mean that the remainder of the compound has the same structure as that of the compound of formula I with a compound of formula VI 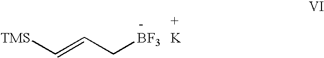
in a solvent selected from the group consisting of water/dichloromethane and water/toluene to form a compound of formula XI 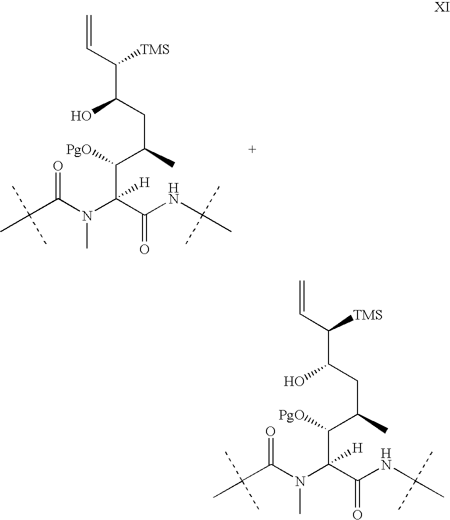
and
(b) converting the compound of formula XI to the cyclosporin A analog of formula I.

22. The process according to claim 21 wherein Pg is an acetyl group.

23. The process according to claim 21, wherein conversion of the compound of formula XI to the cyclosporin A analog of formula I comprises
(b-i) converting the compound of formula XI 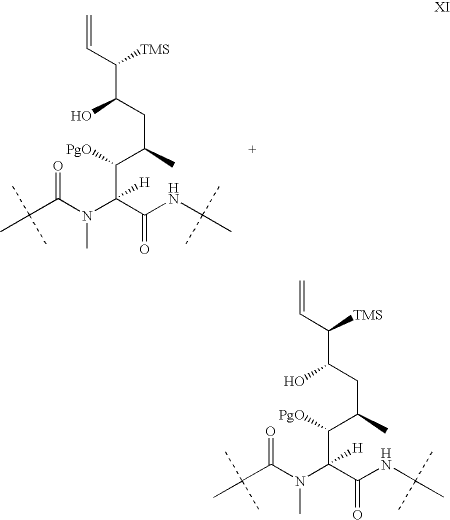
to a compound of formula XII 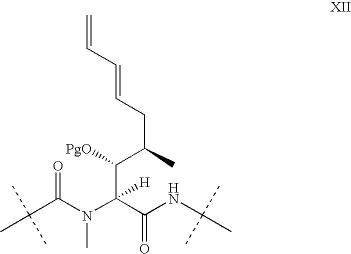
wherein Pg is as defined in claim 21, under acidic conditions, and
(bii) converting the PgO group of the compound of formula XII to a hydroxyl group.

24. A process for the preparation of a cyclosporin A analog of formula I 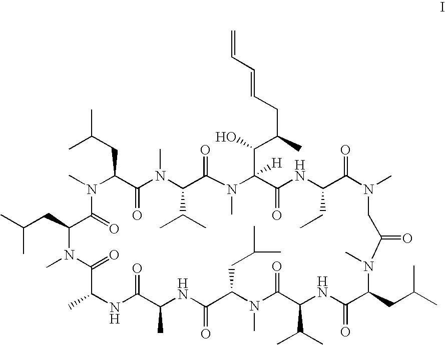
comprising
(a) allylating a compound of formula II 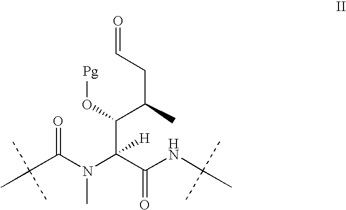
wherein Pg is an acetyl group and the dotted lines mean that the remainder of the compound has the same structure as that of the compound of formula I with a compound of formula VI 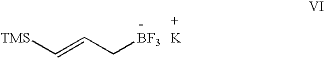
to form a compound of formula XI 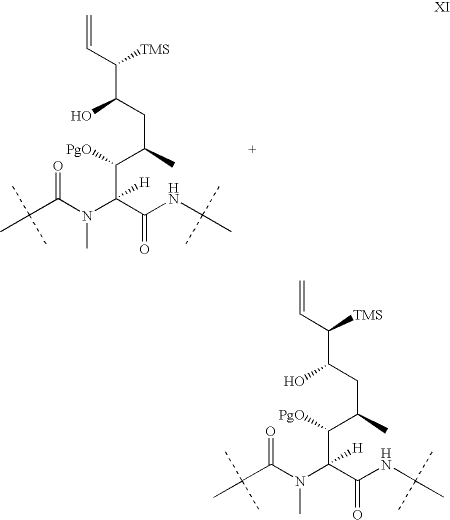
and
(b) converting the compound of formula XI to the cyclosporin A analog of formula I wherein steps (a) and (b-i) are conducted in dichloromethane, tetrahydrofuran or toluene in the presence of BF₃.Et₂O.

25. A process for the preparation of a cyclosporin A analog of formula I 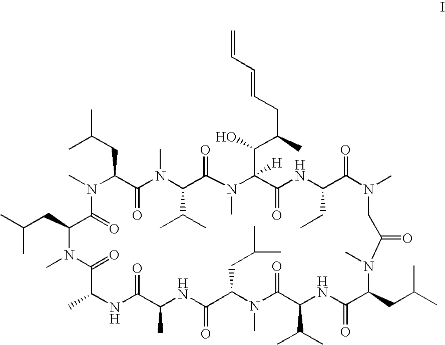
comprising
(a) allylating a compound of formula II 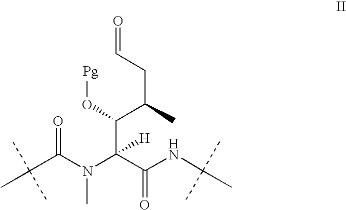
wherein Pg is a protecting group and the dotted lines mean that the remainder of the compound has the same structure as that of the compound of formula I with a compound of formula VII 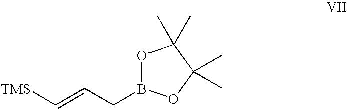
in the presence of BF₃.Et₂O to form a compound of formula XI 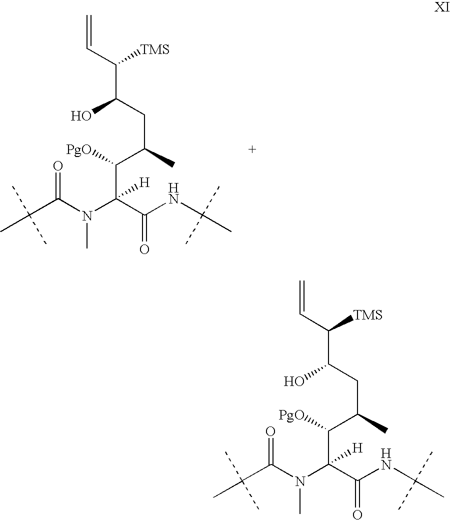
and
(b) converting the compound of formula XI to the cyclosporin A analog of formula I.

26. The process according to claim 25, wherein conversion of the compound of formula XI to the cyclosporin A analog of formula I comprises
(b-i) converting the compound of formula XI 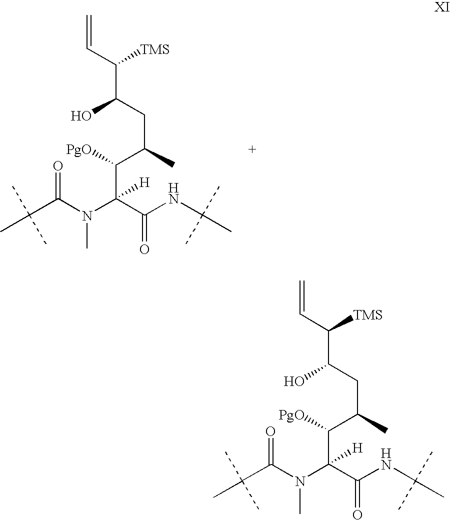
to a compound of formula XII 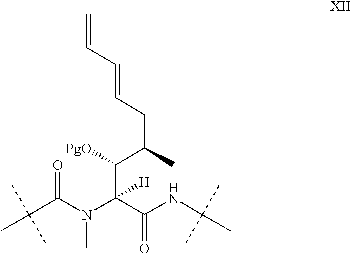
wherein Pg is as defined in claim 25, under acidic conditions, and
(bii) converting the PgO group of the compound of formula XII to a hydroxyl group.

27. The process according to claim 26 wherein steps (a) and (b-i) are conducted in dichloromethane, tetrahydrofuran, or toluene in the presence of BF₃.Et₂O.

28. The process according to claim 25 wherein Pg is an acetyl group.

29. A process for the preparation of a cyclosporin A analog of formula I 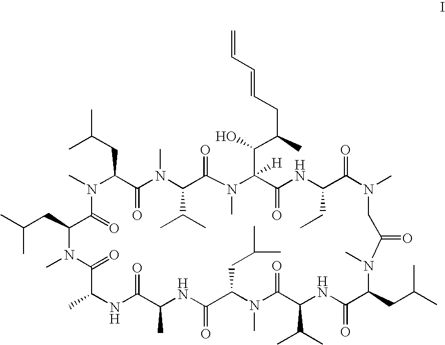
comprising
(a) allylating a compound of formula II 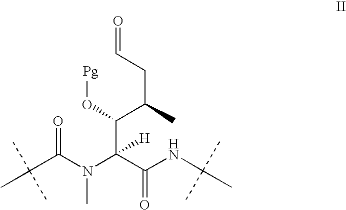
wherein Pg is a protecting group and the dotted lines mean that the remainder of the compound has the same structure as that of the compound of formula I with a compound of formula VII 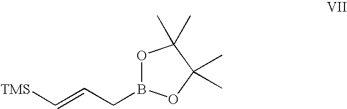
in the presence of formic acid, acetic acid, or a combination of formic acid and acetic acid to form a compound of formula XI 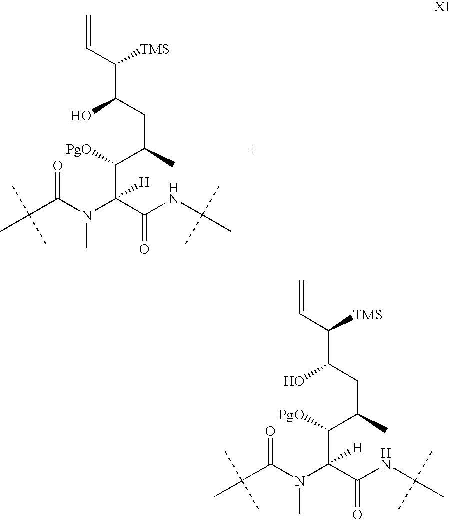
and
(b) converting the compound of formula XI to the cyclosporin A analog of formula I.

30. The process according to claim 29, wherein conversion of the compound of formula XI to the cyclosporin A analog of formula I comprises
(b-i) converting the compound of formula XI 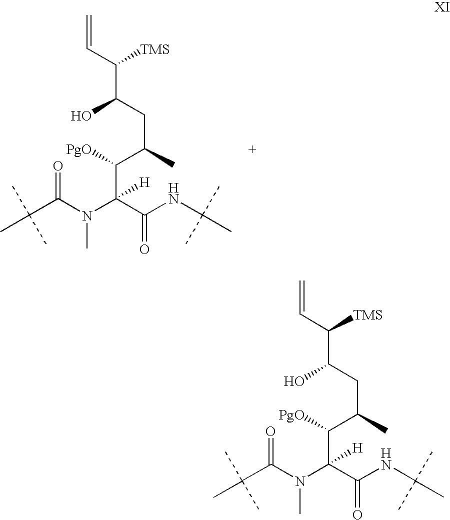
to a compound of formula XII 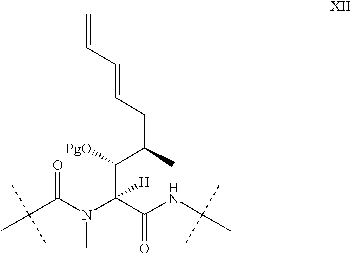
wherein Pg is as defined in claim 29, under acidic conditions, and
(bii) converting the PgO group of the compound of formula XII to a hydroxyl group.

31. The process according to claim 30 wherein steps (a) and (b-i) are conducted in formic acid or acetic acid/formic acid.

32. The process according to claim 31 wherein steps (a) and (b-i) are conducted in a mixture of acetic acid/formic acid and co-solvent selected from dichloromethane, toluene, ethyl acetate and isopropyl acetate.

33. The process according to claim 32 wherein the co-solvent is isopropyl acetate.

34. The process according to claim 30 wherein step (a) is conducted in acetic acid and step (b-i) is conducted by addition of formic acid to the reaction mixture.

35. The process according to claim 29 wherein Pg is an acetyl group.

36. A process for the preparation of a cyclosporin A analog of formula I comprising 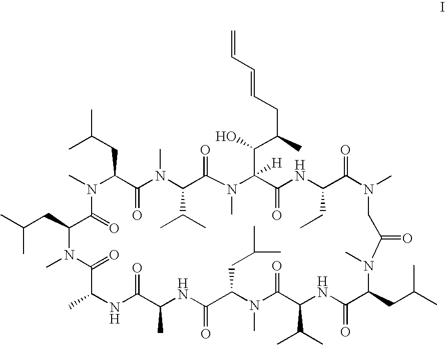
comprising
(a) allylating a compound of formula II 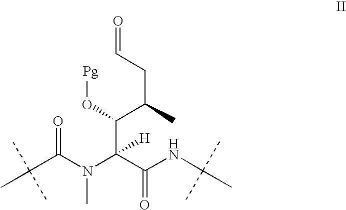
wherein Pg is a protecting group and the dotted lines mean that the remainder of the compound has the same structure as that of the compound of formula I in dichloromethane or toluene with a reaction mixture obtained by a process comprising:
(i) reacting allyltrimethylsilane with butyllithium to form trimethylsilylallyllithium;
(ii) reacting trimethylsilylallyllithium with triisopropylborate or trimethylborate, and then conducting aqueous work up to form a compound of formula XI 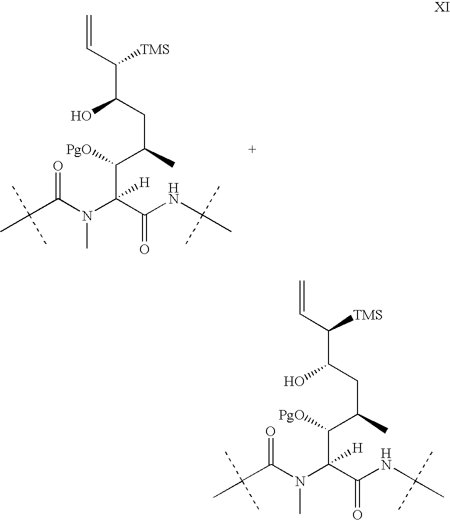
and
(b) converting the compound of formula XI to the cyclosporin A analog of formula I.

37. The process according to claim 36, wherein conversion of the compound of formula XI to the cyclosporin A analog of formula I comprises
(b-i) converting the compound of formula XI 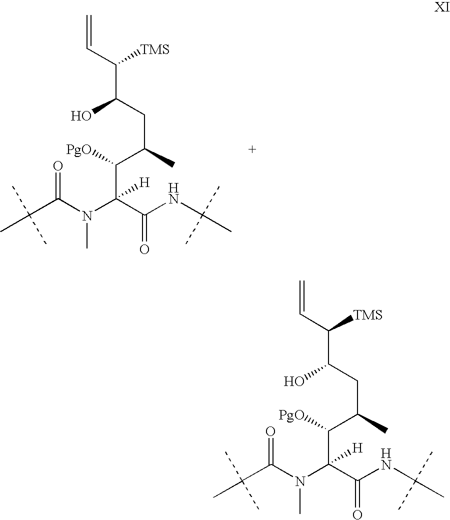
to a compound of formula XII 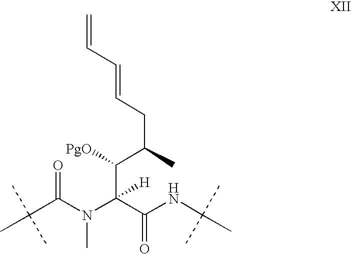
wherein Pg is as defined in claim 36, under acidic conditions, and
(bii) converting the PgO group of the compound of formula XII to a hydroxyl group.

38. The process according to claim 36 wherein Pg is an acetyl group.

39. The process according to claim 36 wherein step (a) is conducted in the presence of tartrates.

40. A process for the preparation of a cyclosporin A analog of formula I 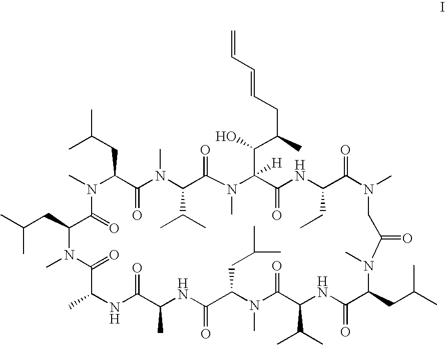
comprising
(a) allylating a compound of formula II 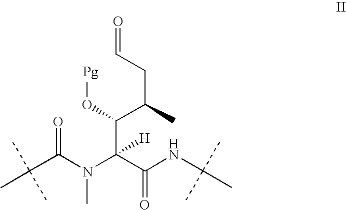
wherein Pg is a protecting group and the dotted lines mean that the remainder of the compound has the same structure as that of the compound of formula I with a reaction mixture obtained by a process comprising:
(i) reacting allyltrimethylsilane with butyllithium to form trimethylsilylallylithium;
(ii) reacting trimethylsilylallylithium with diethylaluminum chloride to form a compound of formula XI 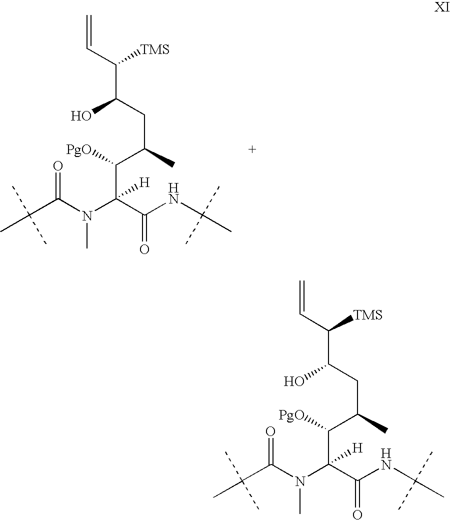
and
(b) converting the compound of formula XI to the cyclosporin A analog of formula I.

41. The process according to claim 40, wherein conversion of the compound of formula XI to the cyclosporin A analog of formula I comprises
(b-i) converting the compound of formula XI 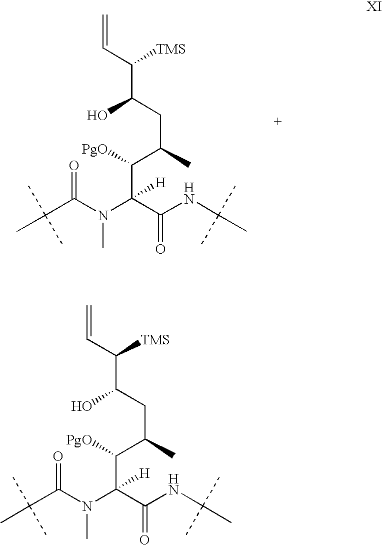
to a compound of formula XII 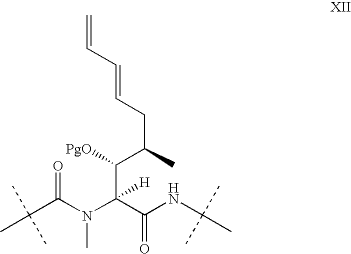
wherein Pg is as defined in claim 40, under acidic conditions, and
(bii) converting the PgO group of the compound of formula XII to a hydroxyl group.

42. The process according to claim 40 wherein Pg is an acetyl group.

43. A process for the preparation of a cyclosporin A analog of formula I 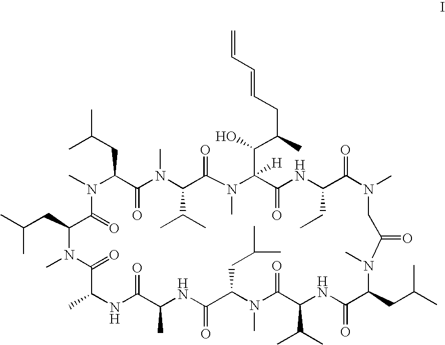
comprising
(a) allylating a compound of formula II 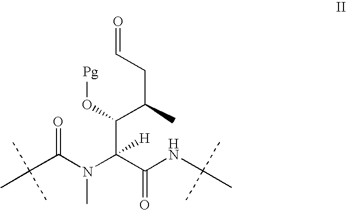
wherein Pg is a protecting group and the dotted lines mean that the remainder of the compound has the same structure as that of the compound of formula I with a reaction mixture obtained by a process comprising:
(i) reacting allyltrimethylsilane with butyllithium to form trimethylsilylallyllithium;
(ii) reacting trimethylsilylallyllithium with titanium tetraisopropoxide or titanium chlorotriisopropoxide
to form a compound of formula XI 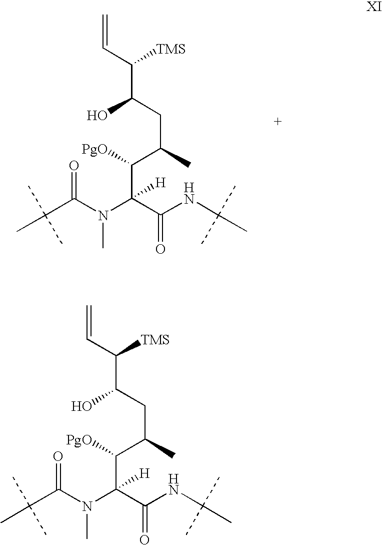
and
(b) converting the compound of formula XI to the cyclosporin A analog of formula I.

44. The process according to claim 43, wherein conversion of the compound of formula XI to the cyclosporin A analog of formula I comprises
(b-i) converting the compound of formula XI 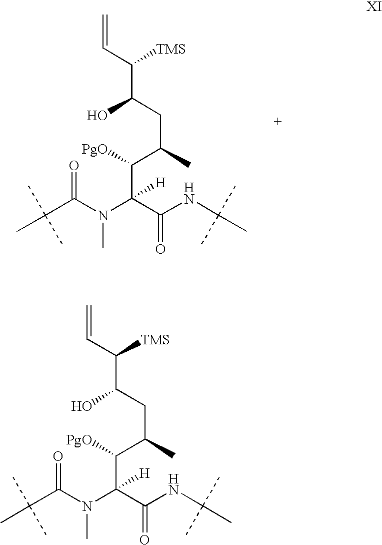
to a compound of formula XII 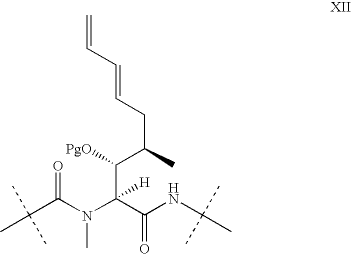
wherein Pg is as defined in claim 43, under acidic conditions, and
(bii) converting the PgO group of the compound of formula XII to a hydroxyl group.

45. The process according to claim 43 wherein Pg is an acetyl group.

46. A process for the preparation of the compound of formula IIIa 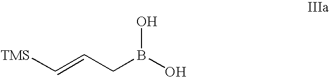
and/or a trimer thereof,
comprising reacting the compound of formula V with water in dichloromethane.

47. A compound of formula V

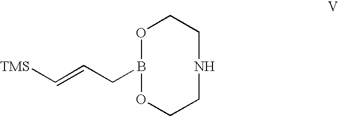

48. A process for the preparation of a compound of formula V, 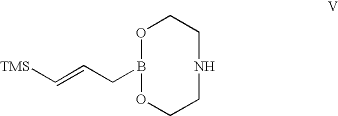
comprising:
i) reacting allyltrimethylsilane with butyllithium to form trimethylsilylallyllithium;
ii) reacting trimethylsilylallyllithium with triisopropylborate or trimethylborate;
iii) conducting aqueous work up; and
iv) reacting the compounds formed in iii) with diethanolamine to form a compound of formula V.

49. A compound of formula VI

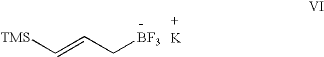

50. A process for the preparation of a compound of formula VI, 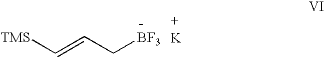
comprising:
i) reacting a compound of formula V 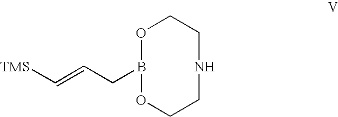
with water to form a compound of formula IIIa 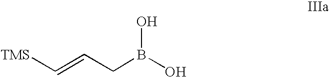
ii) exchanging the solvent of the separated organic phase of the reaction mixture obtained in step i) to methanol, and
iii) reacting a solution of compound of formula IIIa obtained in step ii) with KHF₂ to form a compound of formula VI.

51. A compound of formula IVa [chemical structure]
